(19) United States
(12) Patent Application Publication
(10) Pub. No.: US 2019/0046558 A1
(43) Pub. Date: Feb. 14, 2019

(54) COMPOSITION AND METHODS OF INHIBITING AMYLASE-MEDIATED HYDROLYSIS OF ALPHA (1 to 4)-LINKED GLUCOSE POLYMERS

(71) Applicant: Boston Therapeutics, Inc., Lawrence, MA (US)

(72) Inventor: David Platt, Newton, MA (US)

(73) Assignee: Boston Therapeutics, Inc., Lawrence, MA (US)

(21) Appl. No.: 15/568,935

(22) PCT Filed: May 6, 2016
(86) PCT No.: PCT/US2016/031120

§ 371 Date: Oct. 24, 2017

Related U.S. Application Data
(60) Provisional application No. 62/157,630, filed on May 6, 2015.

(51) Int. Cl. A61K 31/736; A61P 3/10

(52) U.S. Cl. CPC A61K 31/736; A61K 31/702; A61K 2300/00; A61P 3/10

(57) ABSTRACT

PAZ320, a mixture of two galactomannans (GMa and GN/IPβ), is being developed to treat diabetes and inflammatory diseases. Both GMα and GMβ have a β(1->4) mannan backbone, with a high density of α(1->6) linked galactose units. When ingested by diabetic patients, PAZ320 reduces the magnitude of postprandial glucose excursions. PAZ320 functions by binding to enzymes that hydrolyze starch in the gastrointestinal track and thereby reduces steady-state concentrations of low molecular weight sugars like glucose. PAZ320 binds to the α-amylase enzyme from human and porcine sources and thereby attenuates the rate of amylase-mediated hydrolysis of α(1->4)-linked glucose polymers (starch and maltohexaose).

### CROSS REFERENCE TO RELATED APPLICATIONS

This application claims priority to U.S. Provisional Application Ser. No. 62/157,630, filed May 6, 2015, entitled PAZ320 Inhibits Amylase-Mediated Hydrolysis of α(1→4)-Linked Glucose Polymers, which is incorporated by reference herein in its entirety for all purposes.

### BACKGROUND OF THE INVENTION

Starch, an α(1→4)-linked polymer of glucose, is widely found in food, and it is the major component in e.g. bread, potatoes and rice. When food is ingested and starch is digested, this complex carbohydrate is hydrolyzed to various smaller polysaccharides, like dextrin, then on to small sugars like maltotriose and maltose, finally ending up as the monosaccharide glucose. This digestion process generally results in high blood glucose, and in diabetic patients, can lead to hyperglycemia requiring the use of insulin. Persons with diabetes can try to control blood glucose levels by ingesting foods with relatively lower amounts of carbohydrate/starch. However, this may be troublesome in populations where high starch diets are the norm, i.e. in Asia with rice as a dietary staple. It would be quite useful for diabetics therefore to have an agent that could help manage or maintain lower levels of glucose.

In fact, acarbose (Costa & Pifiol, 1997; Scheen, 1998) and voglibose (Dabhi et al., 2013) are two anti-diabetic drugs already in clinical use to treat type 2 diabetes mellitus. Acarbose is a natural microbial pseudotetrasaccharide that binds reversibly and competitively to the oligosaccharide binding site of a-glucosidases, intestinal enzymes that hydrolyze larger carbohydrates like starch and ultimately release glucose. Inhibition of these enzymes reduces the rate of digestion (hydrolysis) of complex carbohydrates, like starch. In this regard, less glucose is absorbed because the carbohydrates are not broken down into glucose molecules. In diabetic patients, the immediate effect is to decrease blood glucose levels. However, acarbose e.g. is generally not sufficiently potent to justify its side effects of diarrhea, flatulence, and in some cases, hepatitis (Lee et al., 2014). Clearly, additional agents need to be developed.

### SUMMARY OF THE INVENTION

PAZ320 is another such agent that is being developed as a dietary supplement to reduce postprandial glycaemia in patients with diabetes. PAZ320 is a mixture of non-glucose-containing complex carbohydrates and is essentially a composite of two galactomannans, one from fenugreek (GMα) and one from guar gum (GMβ), mixed in an approximate 1:4 molar ratio, respectively. Several studies have demonstrated that fenugreek seeds can ameliorate metabolic symptoms associated with type-I and type-2 diabetes in both humans and animals by reducing serum glucose and improving glucose tolerance (Sharma et al., 1990; Gupta et al., 2001), suggesting that fenugreek-derived GMα may be the active component to PAZ320. In a recent clinical study, PAZ320 was reported to reduce glucose levels in about half of the subjects tested (Trask et al., 2013). Although not fully established, the proposed molecular-level mechanism of action of PAZ320 is to block the action of hydrolyzing enzymes that break down carbohydrates, especially starch, into glucose, thereby diminishing the release of glucose into the bloodstream.

As mentioned above, one of the key enzymes that hydrolyzes starch is α-amylase, an enzyme found primarily in saliva and pancreas (Maureen et al, 2000, Voet & Voet, 2005). It is thought that α-Amylase randomly cleaves α(1→4) glycosidic linkages of starch (amylose) to yield dextrin, maltose, or maltotriose, via a double displacement mechanism with retention of anomeric configuration. Glucosidase e.g. then hydrolyzes these saccharides further to glucose. Here, we used NMR spectroscopy to investigate whether GMα and/or GMβ can interact directly with α-amylase and can function to attenuate the rate of starch and maltohexaose hydrolysis.

### BRIEF DESCRIPTION OF THE DRAWINGS

The present disclosure is further described in the detailed description which follows, in reference to the noted plurality of drawings by way of non-limiting examples of embodiments of the present disclosure, in which like reference numerals represent similar parts throughout the several views of the drawings, and wherein:

FIG. 1. shows a colorimetric starch-iodine assay. The effectiveness of GMα (panel A) and GMβ (panel) to inhibit starch hydrolysis (1 mg/ml) mediated by porcine pancreatic α-amylase (1 μM) is shown as a function of the concentration of GMα and GMβ. The Fraction of Reaction Inhibited was calculated using U/ml values determined from the iodine-starch assay as described by (ref), with U/ml=(As62 control−As62 sample)/(As62 starch×20 min×0.1 ml reaction volume), a value that can be interpreted as mg of starch hydrolyzed per minute. The Fraction of “Unfolded” Starch was calculated in the absence of enzyme as A562 taken in the presence of GMα (panel C) and GMβ (panel D) divided by A562 taken in the absence of the GM. General solution conditions are 20 mM potassium phosphate, pH 7, 30° C.;

FIG. 2. shows solution viscosities and effect of glycerol on amylase-mediated hydrolysis of starch. (A) Viscosities shown as cP values for GMα and GMβ were measured as a function of their concentration (mg/ml) as described in the Methods Section. (B) The starch-iodine assay was used with PPA and 1 mg/ml starch to assess the effect of glycerol-mediated solution viscosity. Values for Fraction Reaction Inhibited by glycerol are plotted vs. the viscosity of glycerol. The same plot is shown for Fraction Reaction Inhibited by GMα vs. the solution viscosity of GMα. General solution conditions are 20 mM potassium phosphate, pH 7, 30° C.;

FIG. 3. shows ¹H NMR spectra of starch and starch/GMα with and without amylase. The lower trace shows the ¹H NMR spectrum (3.13 ppm to 4.01 ppm) of starch (1 mg/ml) prior to addition of pancreatic a-amylase (1 μM). The upper trace is for the same starch solution following addition of GMα (4 mg/ml). Starch is a polymer of α(174) linked glucose, that is hydrolytically digested by α-amylase into primarily maltotriose (MT3), maltose (MT2), and glucose (Glc), whose H2 resonances are labeled in the figure. Inserts show the superposition of spectral traces acquired during the time-dependent increase in intensity of MT2, MT3 and Glc H2 resonances as hydrolysis progresses over 11 hours in the absence (lower insert) and presence of GMα (4 mg/ml, upper insert). The increase in intensity reflects the increase in concentration of these saccharides during hydrolysis. General solution conditions are 20 mM potassium phosphate, pH 7, 30° C.;

FIG. 4. shows NMR-derived apparent rates of amylase-mediated hydrolysis. (A) The amount of MT2/MT3 produced during the time course of the amylase-mediated reaction with starch (1 mg/ml) is shown for the initial period of the reaction. Results are shown for starch alone and for starch in the presence of GMα at concentrations of 0.5, 1, 2, and 4 mg/ml, as labeled in the figure. (B) The amount of MT2/MT3 produced during the time course of the amylase-mediated reaction with maltohexaose (1 mg/ml) is shown for the initial period of the reaction. Results are shown for maltohexaose alone and for maltohexaose in the presence of GMα at concentrations of 1 and 2 mg/ml, as labeled in the figure. General solution conditions are 20 mM potassium phosphate, pH 7, 30° C. The concentration of MT2/MT3 produced was determined by using a calibration curve generated by acquiring NMR spectra of maltose (MT2) at known concentrations. The slope of each of these curves effectively provides a measure of the apparent rate of reaction. These values are given in Table 1;

FIG. 5. ¹H NMR spectra of amylase in the absence and presence of GMα. (A) The ¹H NMR spectrum of human salivary amylase (HSA, 50 μM) alone is shown in the bottom trace, and spectra of HSA (50 μM.) in the presence of GMα at 1 mg/ml and 2 mg/ml (top most trace) are shown above this trace. (B) The ¹H NMR spectrum of GMα alone (4 mg/ml) is shown in the bottom-most trace, followed by that of porcine pancreatic amylase (PPA, 50 μM) alone, and then spectra of PPA (50 M.) in the presence of GMα at 0.5, 1, 2, 3, 4 mg/ml (upper most trace). Some resonances that shift during the titration are indicated with arrows. General solution conditions are 20 mM potassium phosphate, pH 7, 30° C.;

FIG. 6. shows ¹H NMR spectra of porcine pancreatic amylase (PPA) in the absence and presence of acarbose (A) and GM (B) are shown. The ¹H NMR spectrum of PPA (50 μM) alone is shown in the bottom trace of each set of spectra. Acarbose was added at concentrations of 1, 10, and 50 μM (upper most trace in A). GM was added at concentrations of at 0.5, 1, 2, and 4 mg/ml (upper most trace in B). Some resonances that shift during the titration are indicated with arrows. General solution conditions are 20 mM potassium phosphate, pH 7, 30° C.;

FIG. 7. shows the X-ray crystal structure of porcine pancreatic α-amylase (PPA) bound with acarbose (PDB access code IDHK) is shown. The X-ray crystal structure of human pancreatic α-amylase (HPA) bound with acarbose (PDB access code 1OSE) is overlaid on the structure of PPA;

FIG. 8 shows a few resonances from the hydrolysis product maltose for free maltose and maltose resulting from reactions with HPA (same as for PPA);

FIG. 9 show the superposition of 1H NMR spectra as maltose is produced from I mg/mL starch in the presence of HPA without GM 1, and then with the enzyme in the presence of starch:GM 1 molar ratios of 1:1 and 1:4;

FIG. 10 show the superposition of 1H NMR spectra as maltose is produced from I mg/mL starch in the presence of HPA without GM 1, and then with the enzyme in the presence of starch:GM 1 molar ratios of 1:1 and 1:4;

FIG. 11 show the superposition of 1H NMR spectra as maltose is produced from I mg/mL starch in the presence of HPA without GM 1, and then with the enzyme in the presence of starch:GM 1 molar ratios of 1:1 and 1:4;

FIG. 12 show normalized overlays of the data the superposition of 1H NMR spectra as maltose is produced from I mg/mL starch in the presence of HPA without GM 1, and then with the enzyme in the presence of starch:GM 1 molar ratios of 1:1 and 1:4;

FIG. 13 show normalized overlays of the data the superposition of 1H NMR spectra as maltose is produced from I mg/mL starch in the presence of HPA without GM 1, and then with the enzyme in the presence of starch:GM 1 molar ratios of 1:1 and 1:4;

FIG. 14 shows the relatively production of maltose in the absence and presence of GM 1;

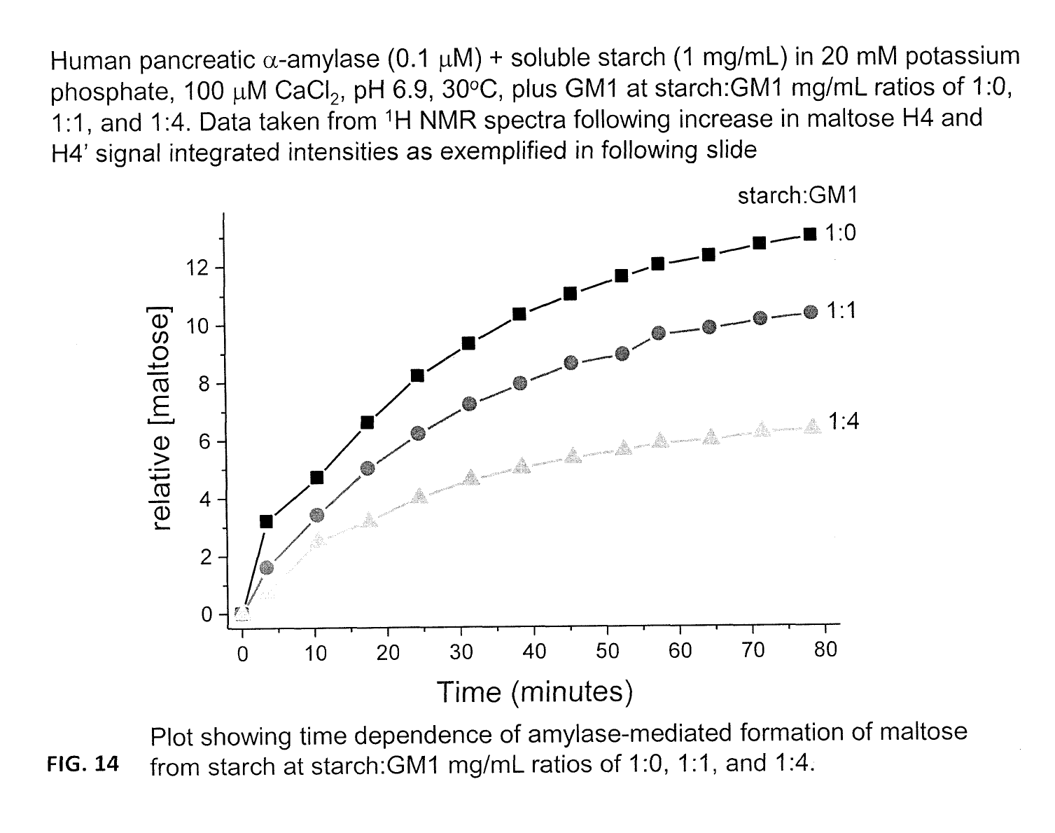

FIG. 15 plots natural logarithm of (1 minus fraction maltose produced) vs time to yield kinetic parameters, i.e. rate constants, k;

FIG. 16 depicts NMR experiments performed using PPA;

FIG. 17 depicts NMR experiments performed using PPA;

FIG. 18 depicts NMR experiments performed using PPA;

FIG. 19 depicts NMR experiments performed using PPA and the depiction of the kinetic data;

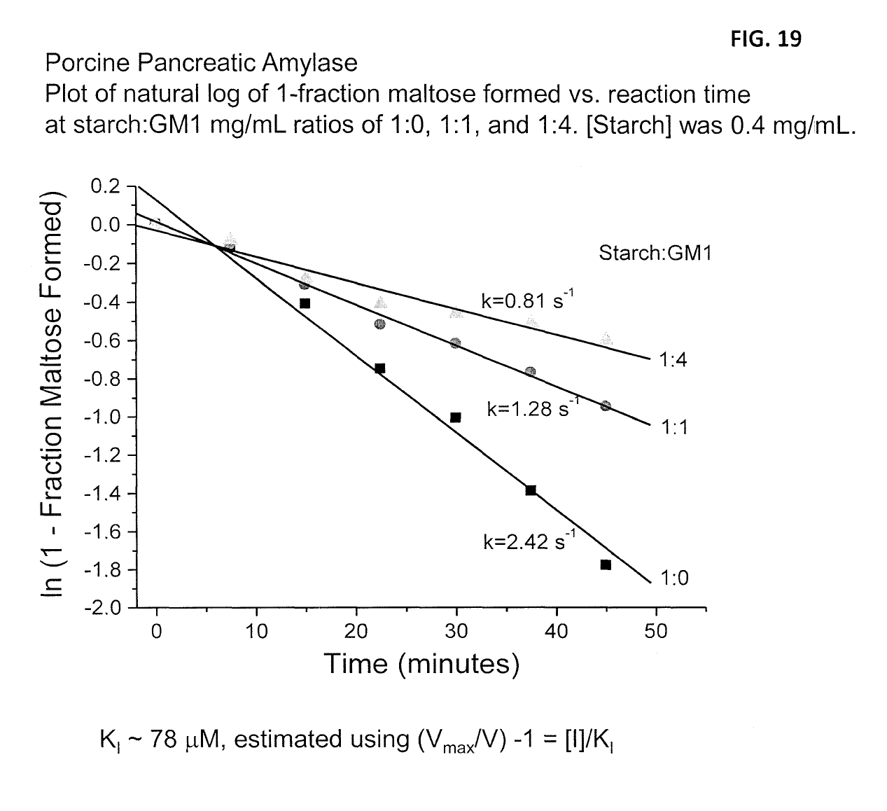

FIG. 20 depicts NMR data indicating that GM 1 indeed binds to the both human salivary amylase and porcine pancreatic amylase;

FIG. 21 depicts NMR data indicating that GM 1 indeed binds to the both human salivary amylase and porcine pancreatic amylase;

FIG. 22 depicts NMR data indicating that GM 1 indeed binds to the both human salivary amylase and porcine pancreatic amylase;

FIG. 23 shows the tryptophan (Trp) region of HAS with its 16 Trp residues as well as that of part of the β-sheet αH region;

FIG. 24 show some NMR data with PPA;

FIG. 25 show some NMR data with PPA; and

FIG. 26 show some NMR data with PPA.

### DETAILED DESCRIPTION OF THE INVENTION

### Amylase-Mediated Hydrolysis of Starch

Embodiments of the technology described herein are based on the discovery that PAZ320, a mixture of two galactomannans (GMα and GMβ), is being developed to treat diabetes and inflammatory diseases when ingested by diabetic patients reduces the magnitude of postprandial glucose excursions. PAZ320 is a composition of at least one purified soluble mannan polysaccharide of high molecular weight and at least one purified mannan polysaccharide of low molecular weight, is more fully described in US Patent Application US 2013/0302471, the contents of which are incorporated by reference in their entirety. It is thought that PAZ320 functions by binding to enzymes that hydrolyze starch in the gastrointestinal track and thereby reduces steady-state concentrations of low molecular weight sugars like glucose. It is further thought that PAZ320 indeed binds to the α-amylase enzyme from human and porcine sources and thereby attenuates the rate of amylase-mediated hydrolysis of α(1→4)-linked glucose polymers (starch and maltohexaose). We further discovered that PAZ320 at 2.5 mg/ml inhibits amylase activity with starch by about 45%, a level of inhibition that is comparable to that from acarbose at 0.13 mg/ml. In addition, we found that the GMα component of PAZ320 is about 5-fold more active than GMβ. Both GMs also act to “unfold” the coiled structure of starch with no effect on their inhibitory potency towards amylase. On the other hand, part of the inhibitory effect from GMα in vitro arises from its effect on increasing solution viscosity. Overall the findings provide insight into how PAZ320 may function in vivo and thereby help patients with diabetes and inflammatory diseases.

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The “or “activate” means an increase of at least 10% as compared to a reference level, for example terms “increased”, “increase” or “enhance” or “activate” are all used herein to generally mean an increase by a statically significant amount; for the avoidance of any doubt, the terms “increased”, “increase” or “enhance an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The colorimetric starch-iodine assay demonstrates the effectiveness of GMα and GMβ to inhibit amylase-mediated hydrolysis of starch. Table 1 gives the rate of starch hydrolysis (mg of starch hydrolyzed per minute) at various concentrations of GMα and GMβ. These data present average values from 4 or 5 experiments with starch concentrations of 1 mg/ml and 5 mg/ml. Results indicate that both GMα and GMβ can inhibit this reaction, with GMα exhibiting a significantly greater effect. With GMα, the rate of hydrolysis with 1 mg/ml starch falls from xx at 0.5 mg/ml to xx at 4 mg/ml. Even at 16 mg/ml of GM/3, the rate is only xx. At the starch concentration of 5 mg/ml, rates are somewhat more attenuated as expected, but nevertheless similar effects are observed.

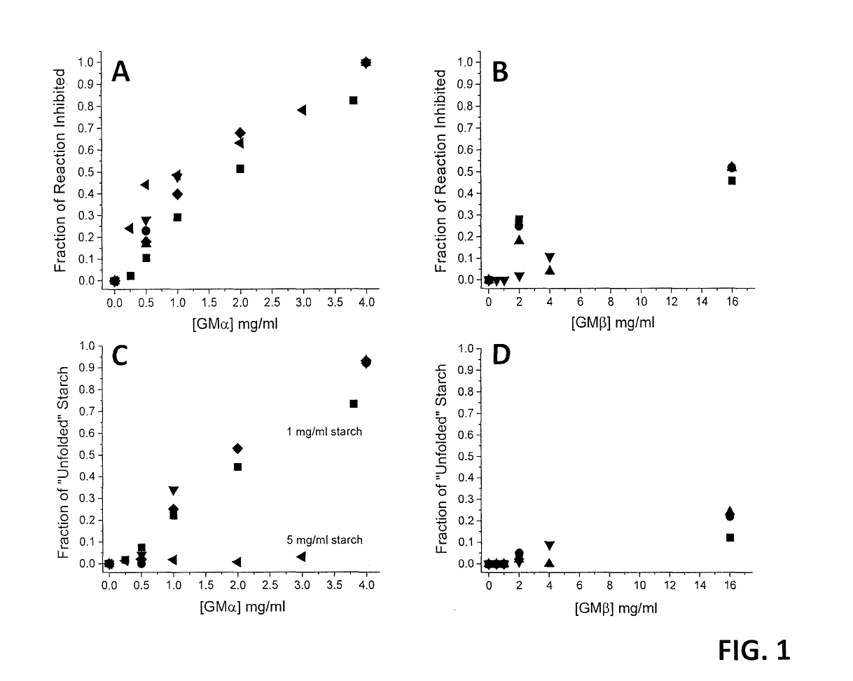

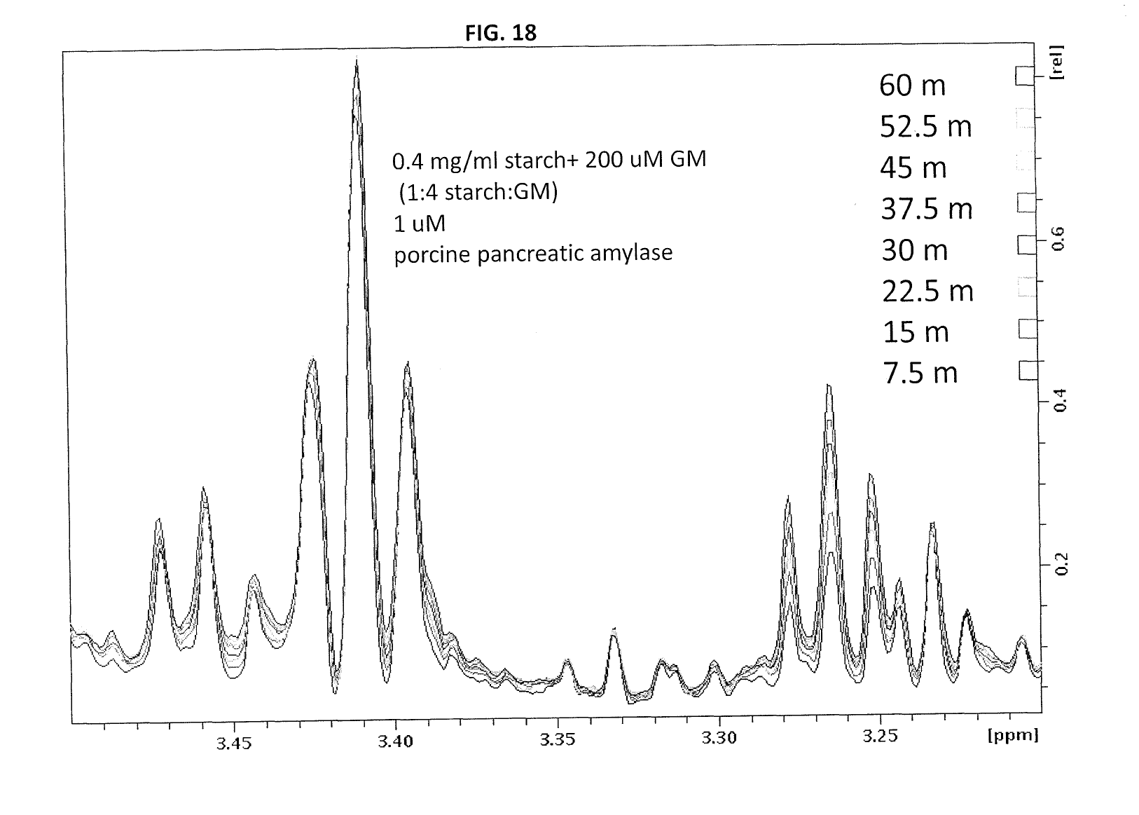

These inhibitory trends are perhaps better appreciated in FIG. 1 which plots the fraction of reaction inhibited (rate with GM divided by rate without GM) vs. the concentration of GMα (FIG. 1A) and GMβ (FIG. 18). Here, we show all individual data points, and not their averages as given in Table I. At 4 mg/ml, GMα appears to inhibit the reaction completely; however, this is somewhat misleading, because the assay is actually at its limit under the specific conditions with which it was performed. Nonetheless, the rate of hydrolysis is clearly dramatically reduced by the presence of 4 mg/ml GMα. On the other hand, the fraction of reaction inhibited with GMβ is only about 0.1 at the same concentration of 4 mg/ml, and is only increased to 0.48 by 16 mg/ml. These data indicate that GMα is about 10 fold more effective than GMβ. Note also that the fraction inhibited by GMα is essentially the same at starch concentrations of 1 mg/ml and 5 mg/ml. Because the concentration of amylase (1 μM) is the same at either starch concentration, this in turn suggests that there is a direct interaction between the amylase and GMα that attenuates the enzyme-mediated hydrolysis reaction.

Because PAZ320 is a combination of GMα and GMβ made up at approximately a 1:4 molar ratio, respectively, we also performed the starch-iodine assay with this combination (Table 1). In this case, the effect appears to be additive. This in turn suggests that GMα and GMβ inhibit amylase activity independently of each other.

We also compared effects from GMα and GMβ with that from acarbose. Table 1 shows that GMα at 2 mg/ml is about as effective as acarbose at 400 μM. Given the weight average MW of GMα (−200 kDa), its concentration is 10 μM. At first glance, this would suggest that GMα is much more effective than acarbose. However, this is misleading, because the binding stoichiometry between amylase and the small pseudotetrasaccharide acarbose is 1:1, whereas for GMα, binding stoichiometry to amylase is unknown, but it is likely to be much greater than 1:1, such that one mole of GMα likely binds more than one mole of amylase.

In the starch-iodine assay, it was found that GMα interacts directly with starch. This conclusion is based on the observation that in the absence of enzyme, the iodine-induced absorbance (A₅₆₂) is significantly lower for 1 mg/ml starch (plus iodine) in the presence of GMα. Iodine intercalates into the starch super-molecular coiled structure to yield an intense blue color. The intensity of the color is related to the amount of starch “folded” structure. In this regard, a reduction in color intensity is directly related to a decrease in the amount of this structure. FIG. 1 plots the fraction of “unfolded” starch as a function of the concentration of GMα (FIG. 1C) and GMβ (Figure ID). We calculated the fraction of “unfolded” starch as A₅₆₂ with GM divided by A₅₆₂ without GM. Note in FIG. 1C that as the concentration of GMα is increased, the fraction of “unfolded” structure of starch is increased significantly, such that by 4 mg/ml GMα, folded structure is near zero. However, as mentioned above, the A₅₆₂ value falls at the limit of the assay, so some structure probably remains at 4 mg/ml, but it is clearly highly attenuated. In this regard, GMα must interact with starch and effectively unfold the coiled structural segments in starch. This also occurs with GMβ, but to a much lesser extent.

To assess whether the unfolding of starch influences results from the assay and/or the effect of GMα on amylase-mediated hydrolysis, we performed the starch-iodine assay at a higher starch concentration (5 mg/ml). At this starch concentration, there is no apparent unfolding of starch (Figure IC), and yet the calculated fraction of enzyme inhibition remains the same (Figure IA). This experiment was important, because the GMα-induced unfolding of starch could have resulted in an artefactual effect as there would be much less color intensity upon addition of iodine (without enzyme), and one would not necessary be able to discern whether it was due to inhibition of the enzyme, unfolding of starch, or some combination of the two. Because the fraction of inhibited reaction is the same at either starch concentration, this concern was alleviated. Further evidence to support this conclusion is provided in the next section with hydrolysis of the smaller and linear α(1→4)-Linked glucose polymer, maltohexaose.

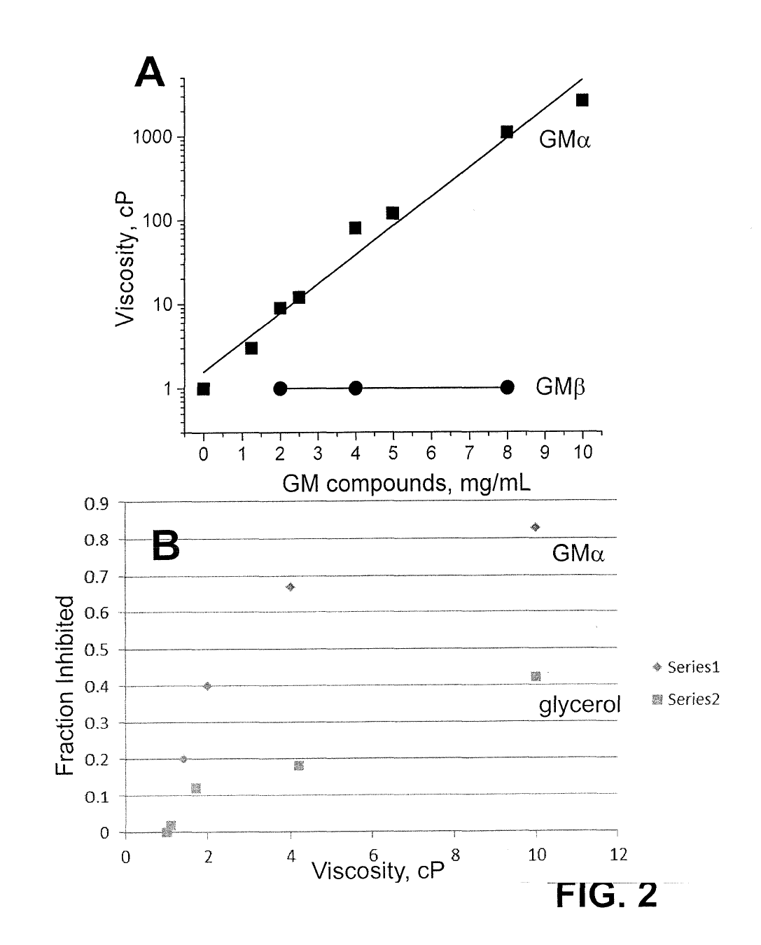

Another concern with GMα and GMβ was their possible effect on solution viscosity. Large polysaccharides like these GMs are known to increase solution viscosity which in turn can attenuate enzyme activity. Therefore, we measured viscosities of our GMα and GMβ solutions, which are plotted in FIG. 2A as viscosity (cP) vs. concentration (mg/ml) of GMα and GMβ. The buffer solution alone had a viscosity of about 1 cP, and solutions of starch at the concentrations used in our experiments (0.4 mg/ml to 5 mg/ml) showed no significant change in that viscosity. The same trend was observed with GMP, where the solution viscosity increased at most to 1.3 cP by 16 mg/ml. On the other hand, the solution viscosity was dramatically increased as the concentration of GMα was increased (FIG. 2A).

To address the question of how GMα-induced changes in viscosity affect amylase activity, we performed the starch-iodine assay as a function of the concentration of glycerol, an agent often used to investigate effects of viscosity on enzyme activity (ref). At 30° C., the viscosity of glycerol solutions remained at or below 1 cP up to about 10% glycerol, and then it increased considerably as the percentage of glycerol was increased further. FIG. 28 plots the fraction of the amylase-mediated hydrolysis reaction inhibited by glycerol vs. the viscosity of glycerol solutions. From these results, it is apparent that increases in solution viscosity do attenuate amylase activity. However, FIG. 28 also plots the fraction of the amylase-mediated hydrolysis reaction inhibited by GMα vs. the viscosity of GMα solutions. Here, it is clear that GMα, at the same viscosity as glycerol, exhibits a considerably greater inhibitory effect than glycerol on amylase activity. Thus only part of the GMα effect on amylase activity arises from GMα-induced changes in solution viscosity. After taking viscosity into account, the inhibitory effect from GMα still stands at about 40% to 50% at 1 mg/ml to 2 mg/ml GMα.

### Amylase-Mediated Starch Hydrolysis Monitored by NMR.

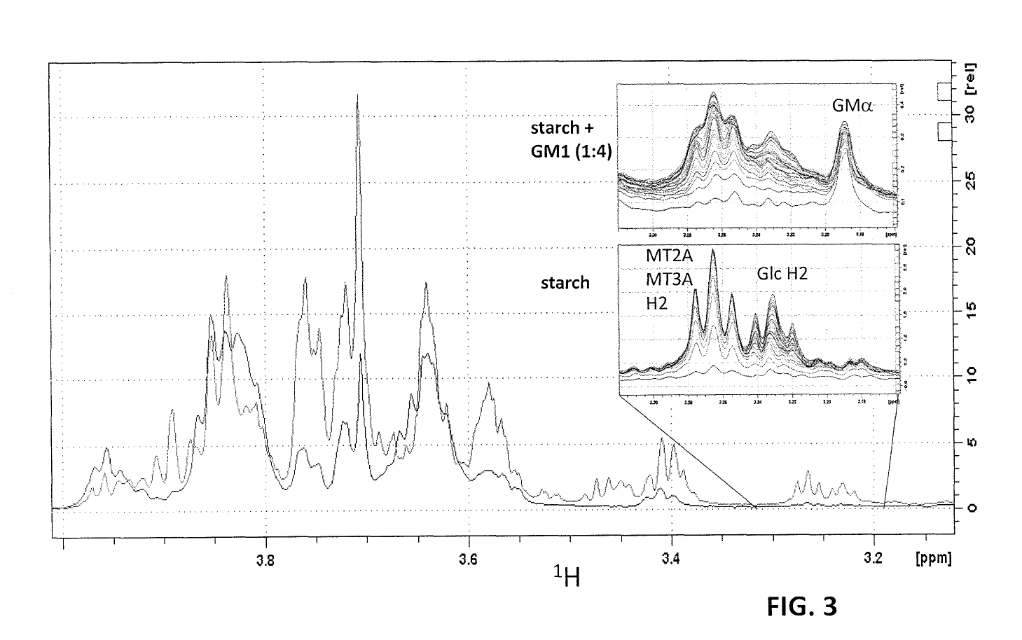

We next used NMR to follow the kinetics of starch hydrolysis and effects from GMα and GMβ. FIG. 3 shows a ¹H NMR spectrum (3.13 ppm to 4.01 ppm) of starch prior to addition of pancreatic a-amylase (lower trace). Starch is a polymer of α(1→4) linked glucose, that is hydrolytically digested by a-amylase into primarily maltotriose (MT3) and maltose (MT2), and then glucose (Glc). The upper spectral trace illustrates what happens to starch following 11 hours in the presence of the enzyme (1 unit). Because all of these saccharides are composed of units of Ole, both spectra appear similar. Upon starch hydrolysis all three of the resulting smaller saccharides (MT3, MT2, and Glc) can be readily identified in a natural abundance ¹³C-¹H HSQC spectrum (data not shown) using ¹H and ¹³C chemical shift assignments previously reported (Goffin et al., 2009).

In the ¹H spectrum shown in FIG. 3, the two apparent triplet resonances (doublets of doublets) at 3.23 ppm and 3.26 ppm are perhaps the best resolved and can be assigned to the H2 resonances of Ole (3.23 ppm), MT2A and MT3A (overlapping at 3.27 and 3.26 ppm, respectively), as labeled. The suffix “A” (e.g. MT3A) refers to the reducing end Glc unit in both MT2 and MT3, whereas B and C refer to the other Glc units towards the non-reducing end of each polysaccharide. The inserts at the right in the figure show the superposition of spectral traces during the time-dependent increase in intensity of these resonances as hydrolysis progresses in the absence (lower insert) and presence of GMα (upper insert). The increase in intensity reflects the increase in concentration of these saccharides during hydrolysis. Since the time intervals between each spectrum in these sets of traces are the same, it should be apparent that production of MT2/MT3/Glc from starch occurs more slowly in the presence of GMα. This observation is consistent with results from the starch-iodine assay that demonstrates the inhibitory effect from GMα on amylase.

As a control, we performed the same NMR experiment with GMα alone plus amylase, and found no apparent change in NMR spectra over time, indicating that amylase does not hydrolyze GMα. This was expected because the GMα polysaccharide is composed of a β(1→4)-linked mannan backbone with a high density of α(1→6)-linked galactose units, and no α(1→4)-linked Glc units. As expected, the same NMR experiment with GMβ yielded the identical result.

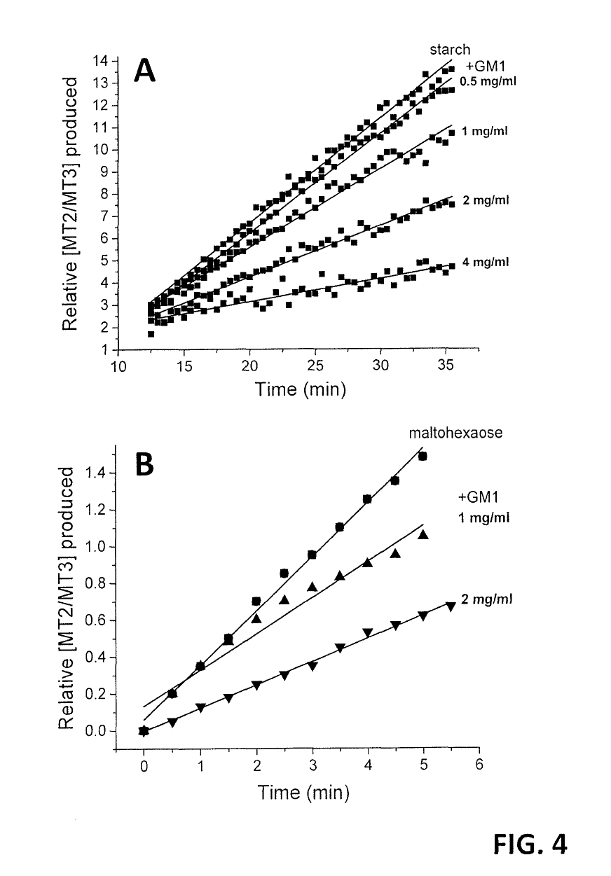

FIG. 4A quantifies our NMR results with starch and GMα by plotting the amount of MT2/MT3 produced over the time course of the hydrolysis reaction for starch alone and for starch in the presence of GMα at concentrations of 0.5, 1, 2, and 4 mg/ml, as labeled in the figure. The concentration of MT2/MT3 produced was determined by using a calibration curve generated by acquiring NMR spectra of maltose (MT2) at known concentrations. Note in FIG. 4A that as the concentration of GMα is increased, the rate of production of MT2/MT3 is significantly decreased, once again indicating the inhibitory potency of GMα on amylase. The slope of each of these curves effectively provides a measure of the apparent initial rate of reaction. These values are given in Table 1. Because the kinetics of amylase-mediated hydrolysis can become complicated by degradation to a number of smaller saccharide products, we have only analyzed and reported changes, i.e. initial velocities, for MT2/MT3 during the start of the reaction.

The trend in the effect of GMα on the rate of hydrolysis parallels that observed in the starch-iodine assay, in that the ratio of rates with and without GMα is essentially the same as the fraction of reaction inhibited with GMα (FIG. 1C). With starch alone, the rate is 29 s⁻¹, whereas in the presence of GMα it is reduced as the GMα concentration is increased: 27 s⁻¹ at 0.5 mg/ml, 22 s⁻¹ at 1 mg/ml, 15 s⁻¹ at 2 mg/ml, and 7 s⁻¹ at 4 mg/ml. In other words, addition of 4 mg/ml GMα reduces the amylase-mediated rate of starch hydrolysis by about 4-fold.

For further assurance that GMα-induced unfolding of starch does not play a significant role in amylase-mediated hydrolysis, we performed the same NMR kinetics experiment using maltohexaose, a linear polymer of α(1→4)-linked Glc units. Maltohexaose is analogous to a small piece of linear starch that cannot form coiled structure. FIG. 4B shows these results plotted in the same way as in FIG. 4A. With maltohexaose alone, the hydrolysis rate is 19 s⁻¹, somewhat slower than with starch. Nevertheless, this rate is also reduced as the GMα concentration is increased: 12 s⁻¹ at 1 mg/ml and 9 s⁻¹ at 2 mg/ml. Because the rates of amylase-mediated hydrolysis of starch and maltohexaose are very much the same, we can conclude that the GMα-induced unfolding of starch does not play a role in amylase-mediated hydrolysis of starch. These results are also consistent with the known mechanism of action of amylase in that it hydrolyzes regions of starch that are not highly organized as coiled structures.

GMα Interacts with a-Amylase.

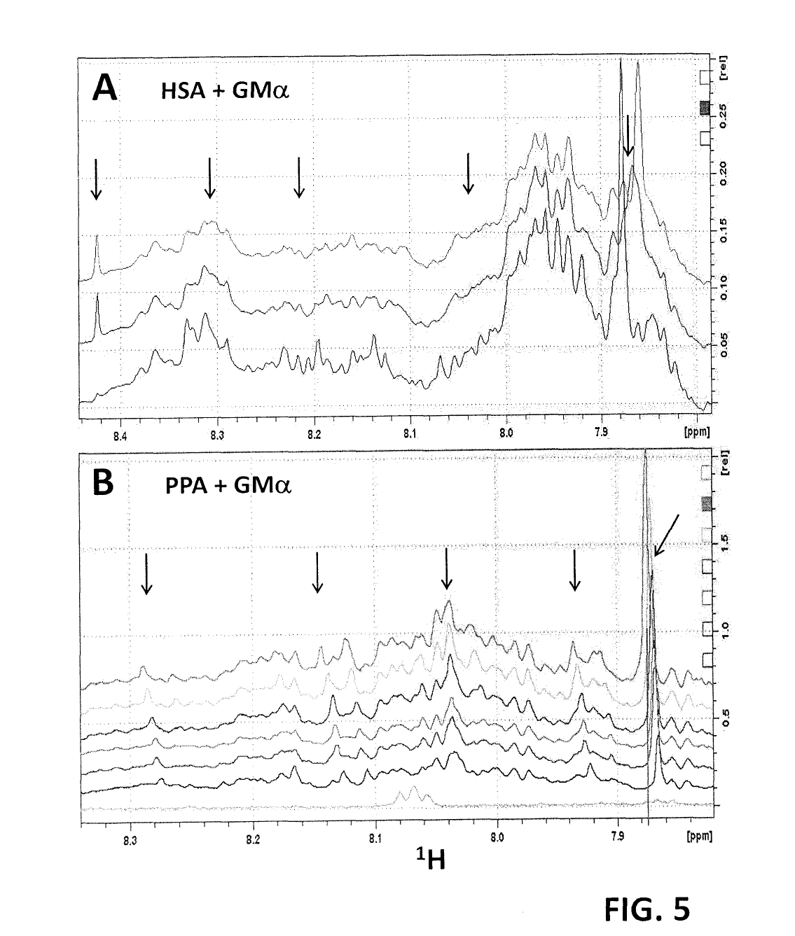

Because our data already suggested a direct interaction between GMα and a-amylase, we used ¹H NMR spectroscopy to assess whether there is indeed a binding event between GMα and amylases from both human pancreatic (HPA), human salivary (HSA) and porcine pancreatic (PPA) sources. Because resonances from GMα overlap up-field (0-6 ppm) with those of amylase, we focused spectral analysis on the downfield region (6-10 ppm). FIG. 5A shows ¹H NMR spectral traces from the NH/aromatic region (7.8 ppm-8.4 ppm) of human salivary a-amylase (50 μM) in the absence (bottom trace) and presence of GMα at 1 mg/ml (middle trace) and 2 mg/ml (top trace). Upon addition of GMα, the a-amylase spectrum is altered with a number of resonances being chemically shifted. The arrows above the spectra indicate a few areas of change.

One resonance at 8.42 ppm may belong to a H2 group of a His residue that becomes more mobile upon addition of GMα. Another relatively sharp resonance at 7.88 ppm may be associated with a HisH4 group; this resonance is significantly shifted (and initially broadened) upon addition of GMα. Aside from shifting resonances, it appears that some resonances may be broadened as the result of an increase in solution viscosity and/or changes in various exchange dynamics. Nevertheless, these data indicate that GMα indeed interacts with the enzyme.

GMα interacts as well with porcine pancreatic a-amylase. FIG. 5B shows ¹H NMR spectra of this enzyme in the absence (second trace from the bottom) and presence of increasing concentrations of GMα from 0.5, 1, 2, 3 to 4 mg/ml (top most trace). Aside from a few resonances around 8.07 ppm that arise from GMα (bottom trace in this figure), all other resonances are associated with those of the enzyme. Once again, as the concentration of GMα is increased, a number of amylase resonances become significantly chemically shifted, some of which are indicated with arrows in this figure. Because site-specific resonance assignments have not been made for either of these amylases, we can only concluded that GMα binds to both amylases, but we lack insight into specifically where on the enzymes GMα interacts.

In both sets of NMR spectra with these amylases and GMα, ¹H resonances shifted steadily during the titrations. This suggests that interactions between these amylases and GMα occur within the fast exchange regime on the chemical shift time scale. Because higher concentrations of GMα were not attainable under the conditions of these NMR experiments, we could not determine equilibrium binding constants from the titrations. However, because interactions occur in the fast exchange regime, the Kd value is likely to be greater than about 20 μM.

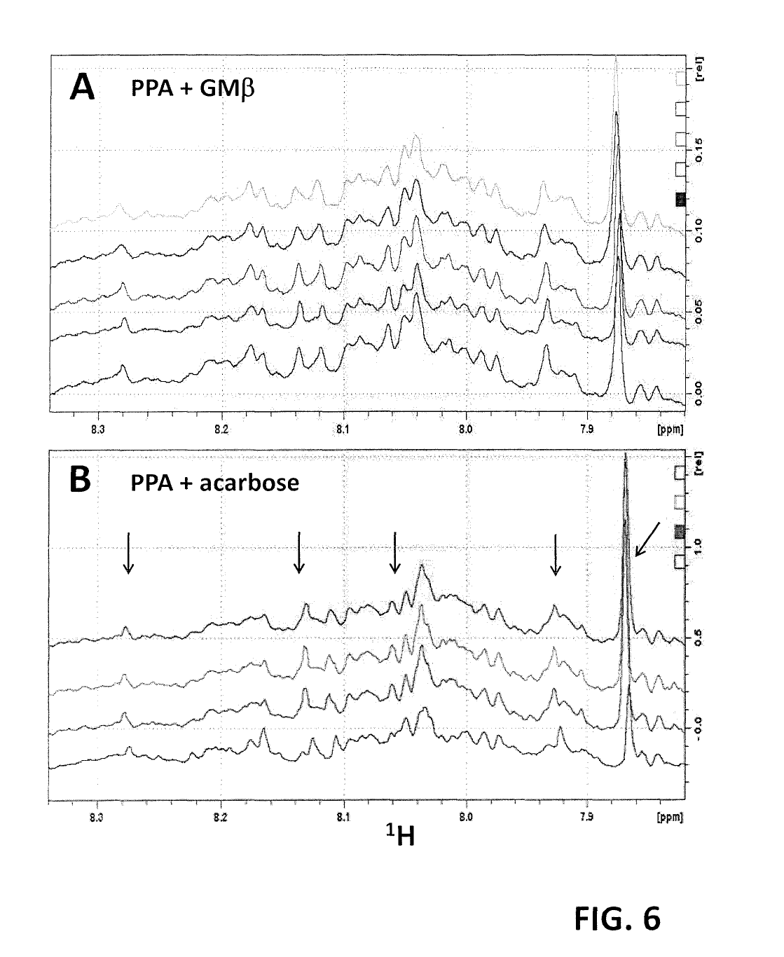

FIG. 6A shows ^{I}H NMR spectra of porcine pancreatic α-amylase in the absence (bottom trace) and presence of increasing concentrations of GMβ from 0.5, 1, 2 to 4 mg/ml. Comparison of these NMR spectra indicates that GMβ at these concentrations has no apparent effect on the enzyme. In other words, GMβ either does not interact with the amylase or interacts only very weakly with the amylase, unlike GMα. This observation is consistent with GM up to 4 mg/ml eliciting only weak, if any, inhibitory effect on amylase function.

Acarbose is a well-known, anti-diabetic drug that interacts with amylase as a competitive inhibitor. For this reason, we also acquired ¹H NMR spectra of a-amylase in the presence of acarbose (1 μM, 10 μM, and 50 μM). FIG. 6B shows the series of these NMR spectra, with the spectrum of amylase alone at the bottom, followed by the spectra of amylase in the presence of these acarbose concentrations. Spectral changes are essentially complete upon addition of 1 μM acarbose, suggesting that the equilibrium dissociation constant, Kd, for binding of acarbose to this amylase is below 1 μM, an observation that is consistent with the Kd value reported. Note that many of those resonances shifted by addition of acarbose are the same as those shifted by the presence of GMα (FIG. 5B). While not allowing for any definitive conclusion, this comparison does suggest that GMα may interact with the enzyme in a similar fashion as acarbose.

Our results demonstrate that PAZ320 effectively inhibits amylase-mediated hydrolysis of α(1→4)-linked glucose polymers (starch and maltohexaose). Of the two galactomannan polysaccharides (GMα and GMβ) that comprise PAZ320, GMα is the primary agent that promotes this activity. Even taking into account the effect from GMα-mediated increases in solution viscosity, GMα is about 5-fold more effective than GMβ at the same concentration.

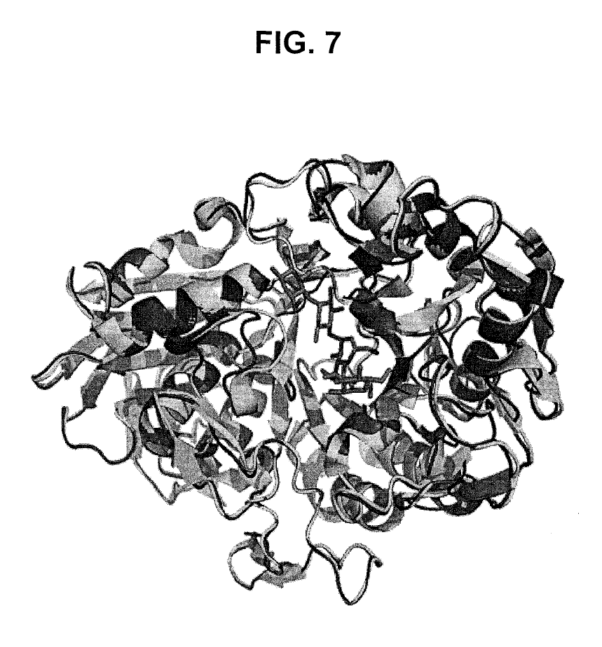

On the molecular level, we found that GMα directly interacts with a-amylase from both human and porcine sources, an event that likely mediates the inhibitory potency of PAZ320. As shown in the literature, α-amylases contain a number of distinct structural domains (e.g. Ramasubbu et al., 1996; Kadziola et al., 1998). The catalytic domain has a structure consisting of an eight-stranded α/β barrel that contains the active site, interrupted by an approximate 70-amino acid residue calcium-binding domain, and a carboxyl-terminal Greek key P-barrel domain. FIG. 7 shows the X-ray crystal structure of porcine pancreatic α-amylase (PPA) (PDB access code 1 DHK), overlaid with that of human pancreatic a-amylase (HPA) bound with acarbose (PDB access code 1OSE). While our NMR data do not report on exactly where on the structure of the amylase GMα interacts, it appears that residues from a P-sheet region(s) of the enzyme are most perturbed by the binding event. Moreover, because the same set of resonances appears to be affected by binding of acarbose, they may share the same binding region. The binding region of acarbose does lie within a P-sheet domain at the active site of the enzyme. Without being bound to any particular theory, we propose that GMα binds around the same region. In addition, our NMR data also suggest that GMα binding to amylase is specific and may conformationally stabilize parts of the structure of the enzyme.

Acarbose binds amylase very strongly, with a Ka value in the nano-molar range. On the other hand, we find that GMα binds amylase with a Ka that appears to fall in the high micro-molar range. Nevertheless, PAZ320, like acarbose, is effective in vivo. Recently, a successful clinical study was reported on PAZ320 that demonstrated reduction in the magnitude of 2-hour postprandial glucose excursions in individuals with type 2 diabetes. Interestingly, in that clinical study, PAZ320 was administered via ingestion at doses of 8 g and 16 g per patient. If one uses blood volume in humans (about SL) to calculate PAZ320 concentration in mg/ml, it comes out to be about 1.6 to 3.2 mg/ml upon ingestion. This is essentially the same range that we used in our biophysical studies in which we demonstrated in vitro that PAZ320 (i.e. GMα) effectively inhibits enzyme-mediated hydrolysis of starch. Now, of course, when one ingests PAZ320, it moves undigested through the GI track, where volumes are less clear, and some starch is digested by the presence of amylase in saliva.

In this clinical study, about 50% of subject patients were not responsive to PAZ320 treatment. One possible reason for this could be related to the fact that PAZ320 was only consumed 2-hours prior to assessment of postprandial glucose excursions. In our biophysical studies, we observed optimal GMα-mediated inhibitory effects on amylase when solutions of starch and GMα (and GMβ) were allowed to mix overnight prior to us. In this regard, there is an apparent kinetic phase during which GM interacts with starch polymer. In the clinical study, there may have been more responders were the study performed when the GM-containing meal was ingested about 24 hours in advance prior to screening. Overall, our results provide insight into the mechanism of action of PAZ320 on the molecular level and its use as a therapeutic agent in the treatment of diabetes.

The efficacy of a given treatment for diabetes can be determined by the skilled clinician. However, a treatment is considered “effective treatment,” as the term is used herein, if any one or all of the signs or symptoms of blood glucose levels are altered in a beneficial manner or other clinically accepted symptoms are improved, or even ameliorated, e.g., by at least 10% following treatment with an agent as described herein. Methods of measuring these indicators are known to those of skill in the art and/or described herein.

An effective amount for the treatment of a disease means that amount which, when administered to a mammal in need thereof, is sufficient to result in effective treatment as that term is defined herein, for that disease.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### Examples

This invention is further illustrated by the following examples, which should not be construed as limiting. An experiment was undertaken to assess whether GM I interacted with human salivary amylase and porcine pancreatic amylase, 1H NMR spectra were acquired on the enzyme in the absence and presence of varying concentrations of GM 1, and perturbations to 1H resonances arising from the amylase protein were followed upon addition of GM I.

Production of small sugar molecules from e.g. hydrolysis of starch can have harmful effects on the body, e.g. high blood glucose levels that can be especially problematic in diabetic patients, and higher cholesterol levels that can promote atherosclerosis, heart attack and/or stroke. The addition of fiber to the diet has been shown to decrease glucose and cholesterol levels, as well as to increase fecal bulk resulting in a potential decrease in colon cancer, and to increase the level of dietary satiety for better weight control.

Galactomannan (GM) is a viscous, soluble fiber that reduces post-prandial glucose, cholesterol, triglycerides and insulin levels in the blood. Although the mechanism of action is not entirely understood, the most commonly stated hypotheses are based upon the increase of viscosity of digestive contents, such as slowed gastric emptying/flow through the intestine, decrease in peristalsis, decrease contact of intestinal walls with digesta, decreased absorption, and slowed movement of substrates and enzymes.

A GM-based product (PAZ320) further described in US Patent Application No. 2013/0302471, the contents of which are incorporate by reference. The rationale for this experiment was to use biophysical methods, primarily NMR spectroscopy, to assess the effects from GM I (apparent active ingredient) and additives (GM2) on carbohydrate hydrolyzing enzymes. It was found that GM I binds to human and porcine pancreatic amylases, as well as to human salivary amylase, and 2) binding of GMI to these enzymes significantly reduces the rate of starch hydrolysis into smaller sugar units, e.g. maltose.

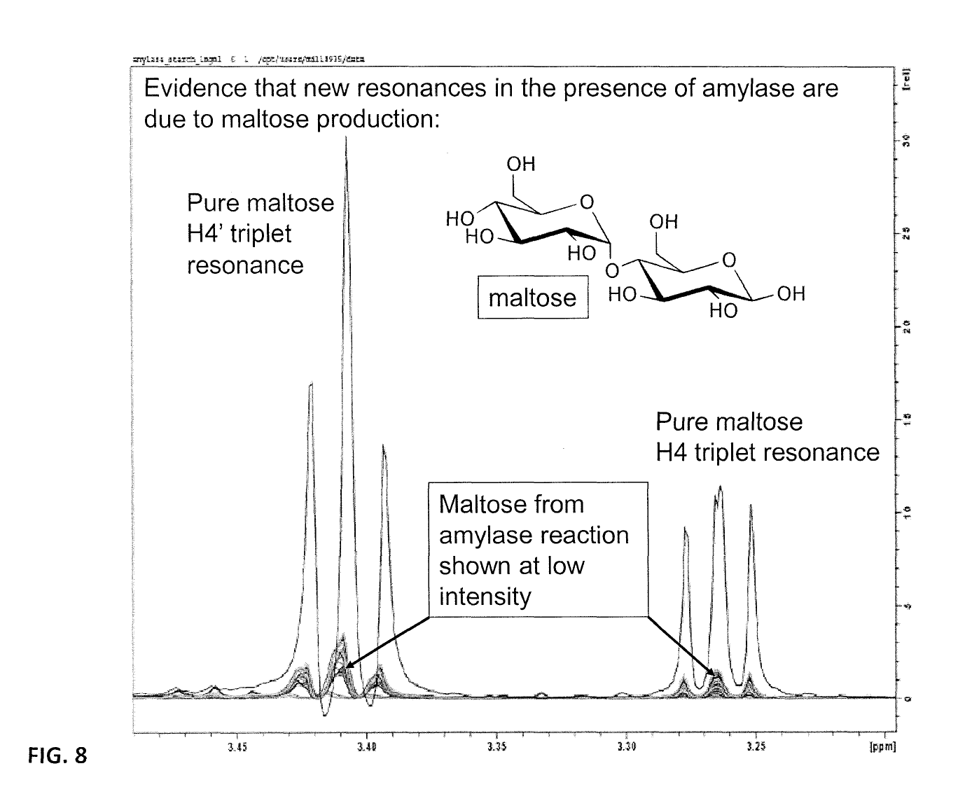

GMl Effect on Amylase-Mediated Hydrolysis of Starch. To assess the effects of GM I on the amylase-mediated hydrolysis of starch, human pancreatic amylase (HPA) and porcine pancreatic amylase (PPA) (0.1 μM, I O units of either) were used with soluble starch (I or 0.4 mg/mL, respectively) and the kinetics of formation of product maltose were followed in the absence and presence of GM I at starch:GM I molar ratios of 1:0, I:I, and I:4. 1H NMR spectra were acquired at time points from O to −80 minutes, and intensities of H4 and H4′ resonances from product maltose were monitored, as exemplified in FIG. 8 that shows a few resonances from the hydrolysis product maltose for free maltose and maltose resulting from reactions with HPA (same as for PPA). We monitored these resonances from maltose because there was no overlap with resonances from GMl or the enzyme.

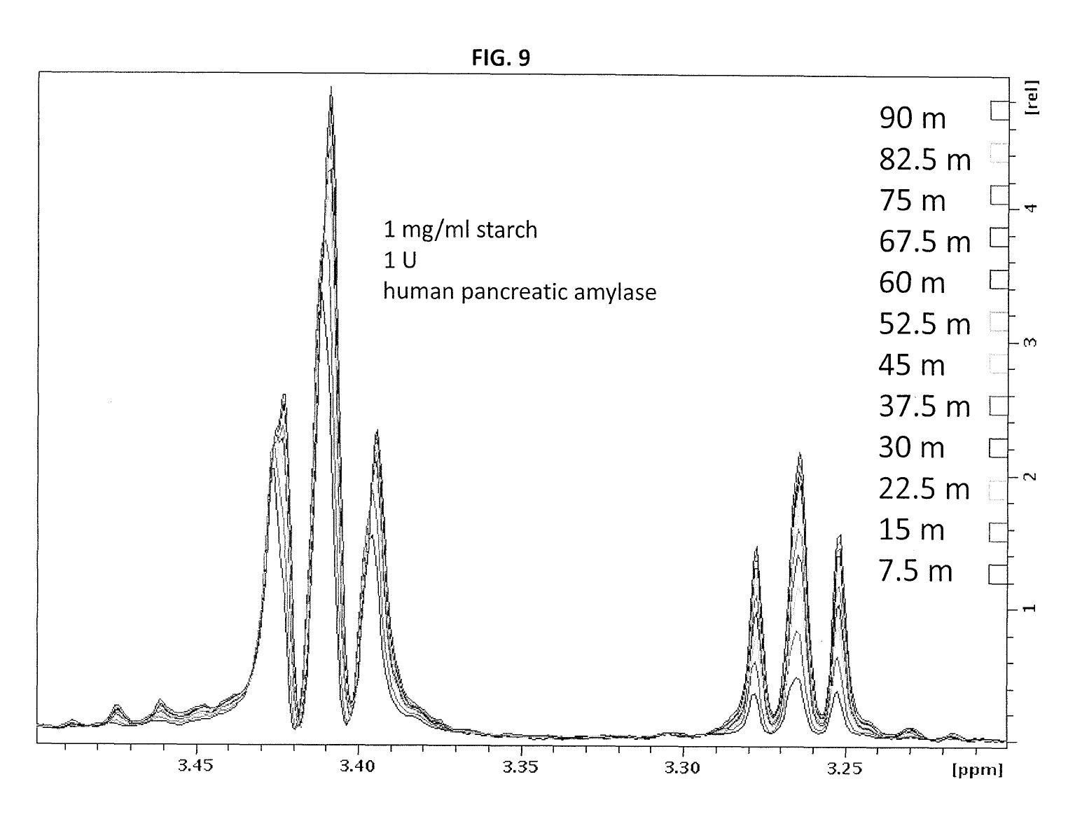

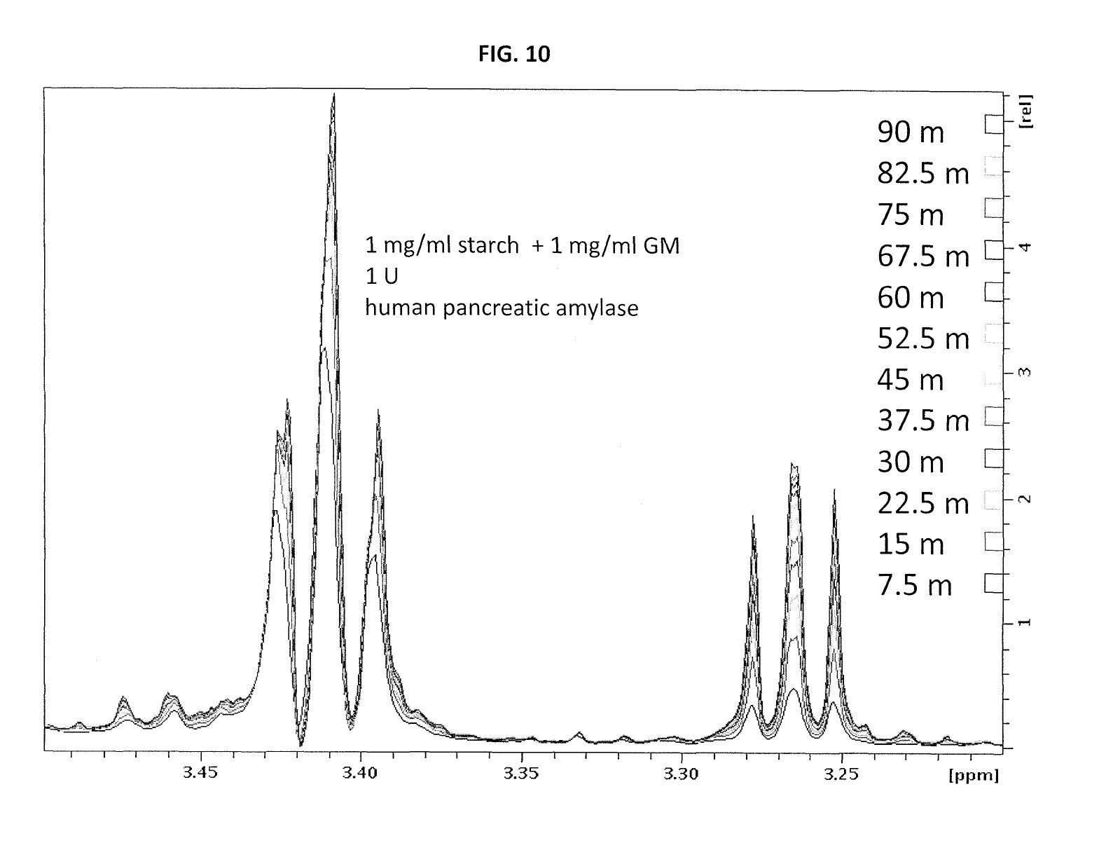

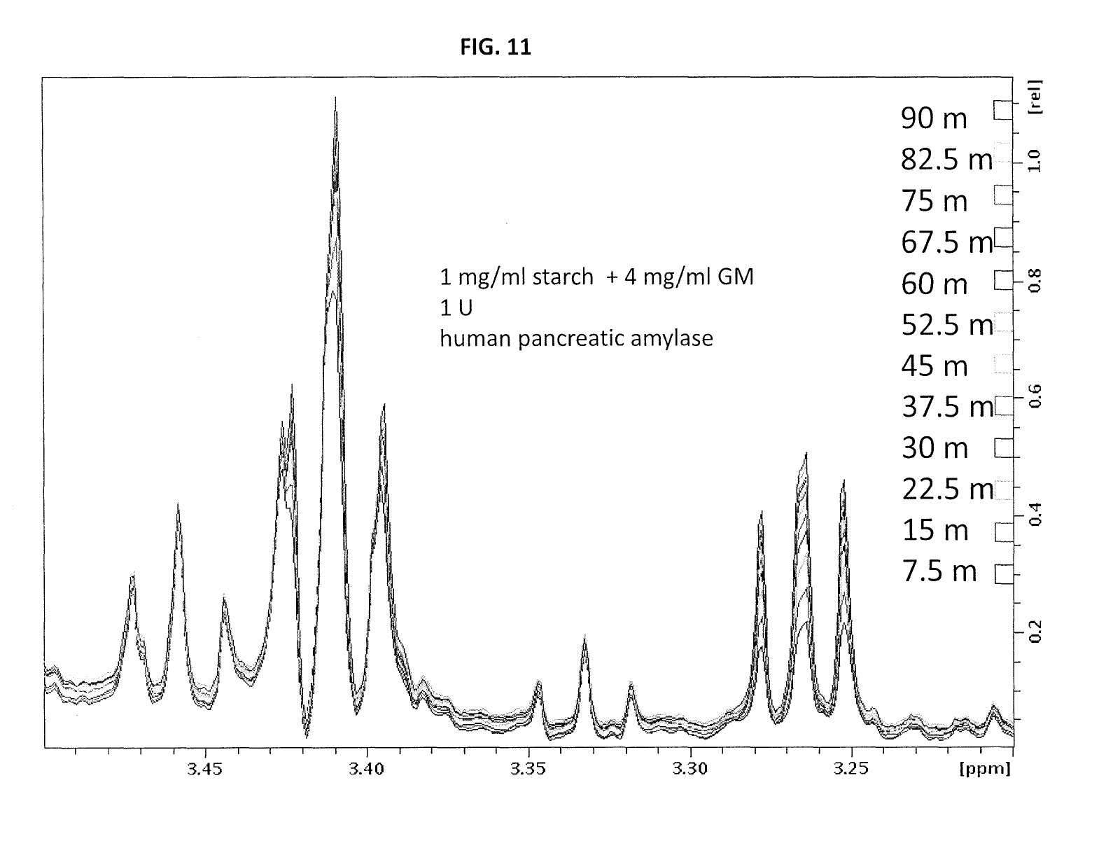

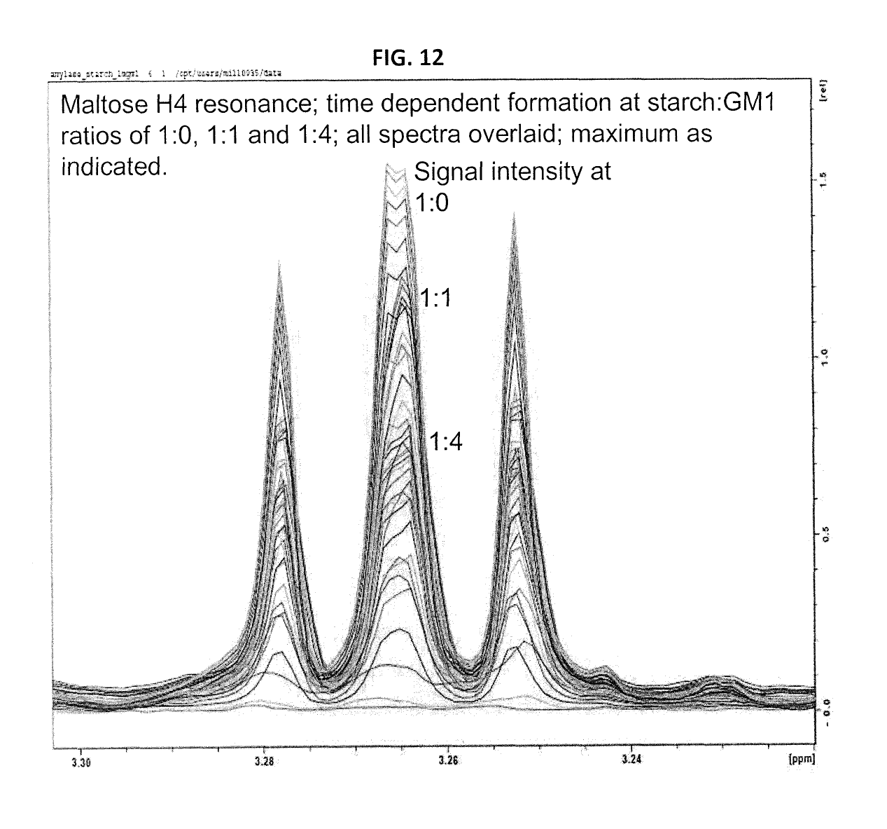

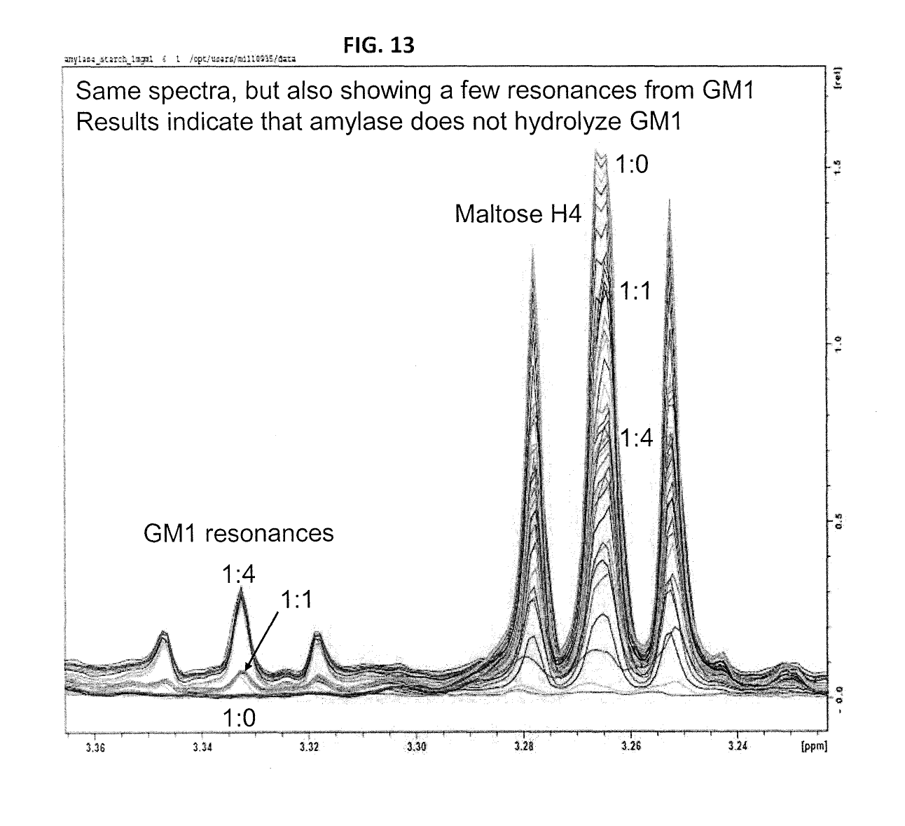

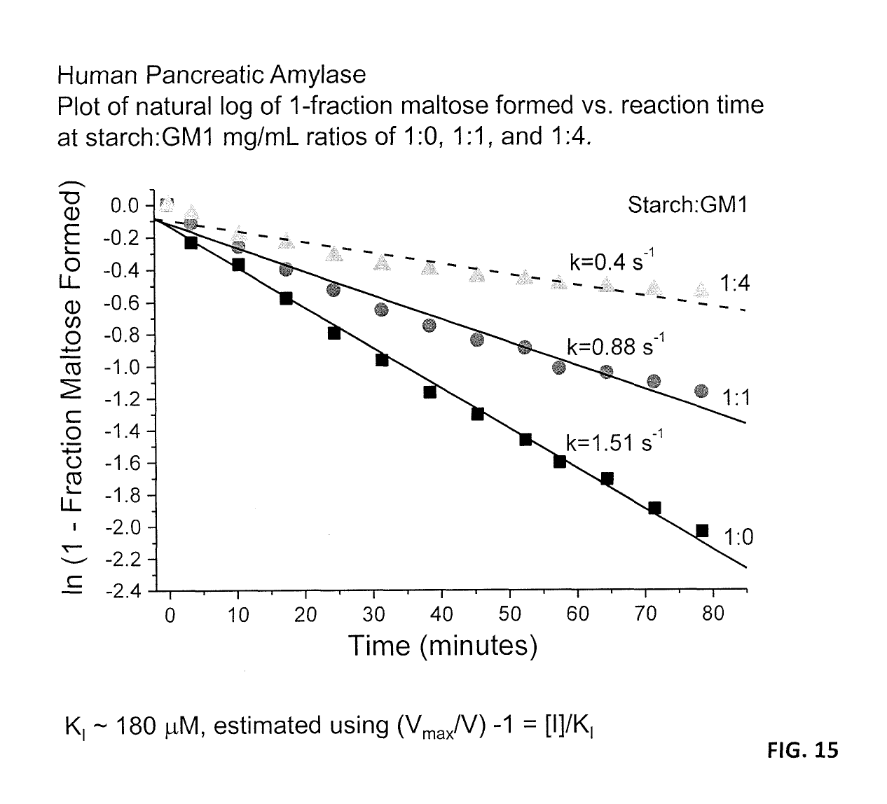

FIGS. 9, 10 and 11 show the superposition of 1H NMR spectra as maltose is produced from I mg/mL starch in the presence of HPA without GM 1, and then with the enzyme in the presence of starch:GM 1 molar ratios of 1:1 and 1:4. The relative effects are better appreciated in FIGS. 12 and 13 which show normalized overlays of the data. Here it is apparent that GM 1 significantly slows production of maltose from starch as shown in slide 14 that shows the relatively production of maltose in the absence and presence of GM 1, and FIG. 15 plots natural logarithm of (1 minus fraction maltose produced) vs time to yield kinetic parameters, i.e. rate constants, k. Note that there is about a 4-fold reduction in the rate of hydrolysis at the starch:GM I molar ratio of I:4.

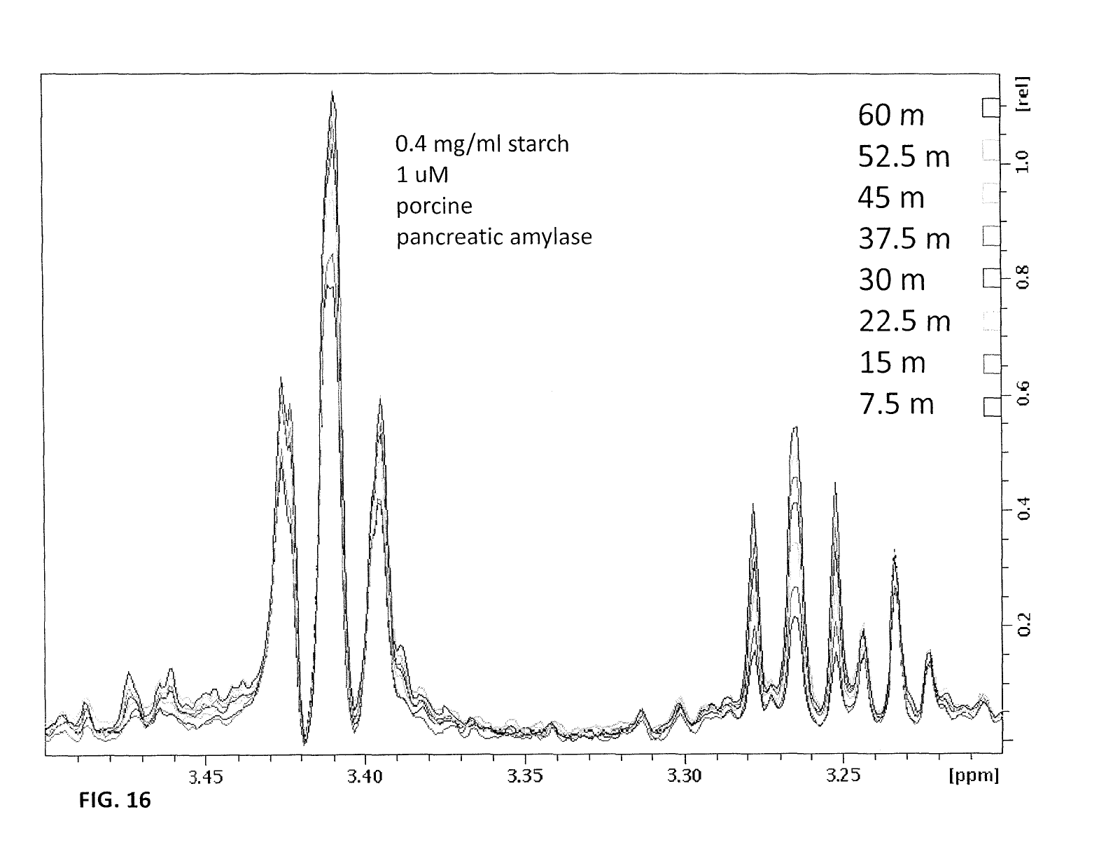

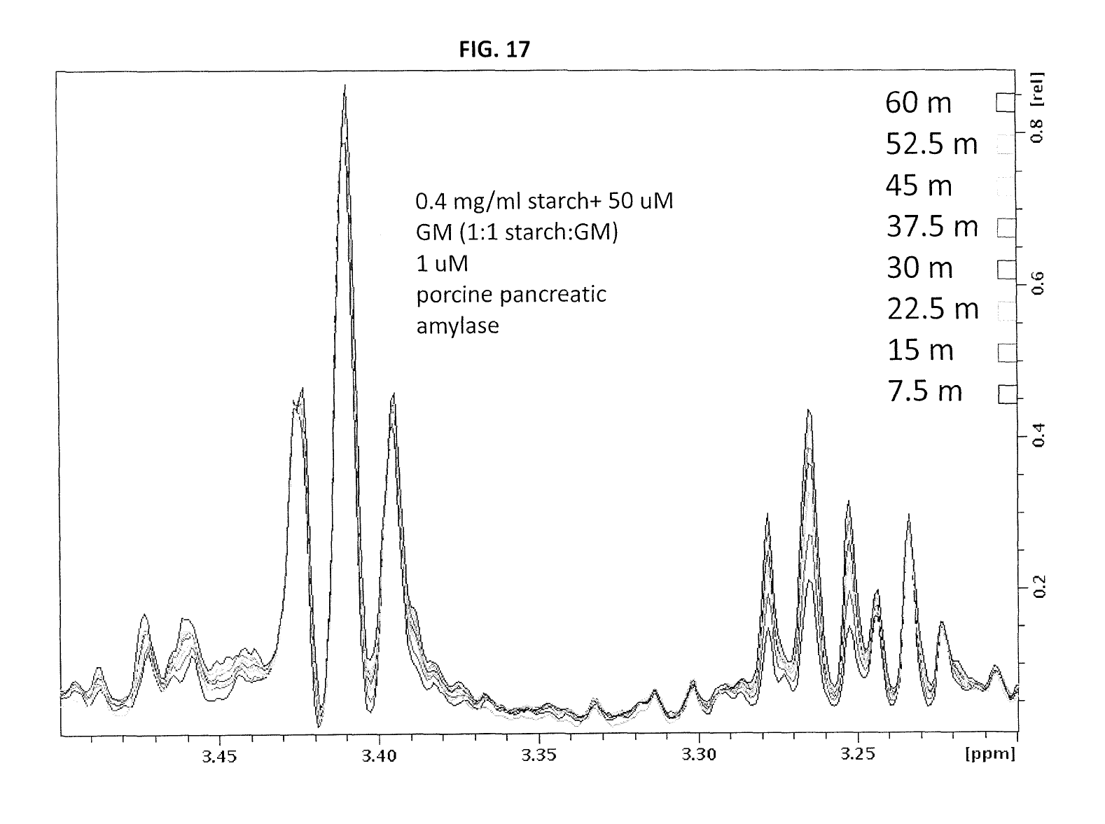

The same NMR experiments were then performed using PPA, as shown in FIGS. 16, 17 and 18. Analysis of these kinetic data is shown in slide 19. Note here that rate constants are somewhat greater with PPA. Nonetheless, the trends are clearly the same as with HPA. Note also that guesstimated K, values are slightly different. K, for PPA is about 78 μM, where that value is somewhat larger at about t 80 μM with HPA. This would be consistent with tighter binding of GM I to PPA than to HPA. As a control, we also added HPA and PPA to GMl alone, and nothing was produced, i.e. GMl is not hydrolyzed by these enzymes, as expected.

To assess the effects of GM I on the amylase-mediated hydrolysis of starch, human pancreatic amylase and porcine pancreatic amylase (0.1 μM, 10 units of either) were used with soluble starch (0.4 or 1 mg/mL) and the kinetics of formation of product maltose were followed in the absence and presence of GMl at starch:GMI molar ratios of 1:0, 1:1, and 1:4. 1H NMR spectra were acquired at time points from O to −80 minutes, and intensities of H4 and H4′ resonances from product maltose were monitored. Kinetic parameters were determined by plotting the natural logarithm of (1 minus the fraction of maltose produced) vs. time. The slope of this plot essentially yields the rate constant, k in units of inverse time (min-1 or s-1). Kr values were crudely estimated by using the equation (Vmax/V)−1=[I]/K1, where k values were used instead of V values.

Results & Discussion

GMl Binding to HSA and PPA. Initial ¹H NMR data attempted to use 2D NMR spectra, but experiments appeared to be inconsistent due to limited quantity of enzyme, resulting in relatively poor signal to noise. Instead, ID 1H NMR spectra were acquired with HSA and PPA in the absence and presence of varying concentrations of GM I, and perturbations to 1H resonances arising from the amylase proteins were followed upon addition of GM 1.

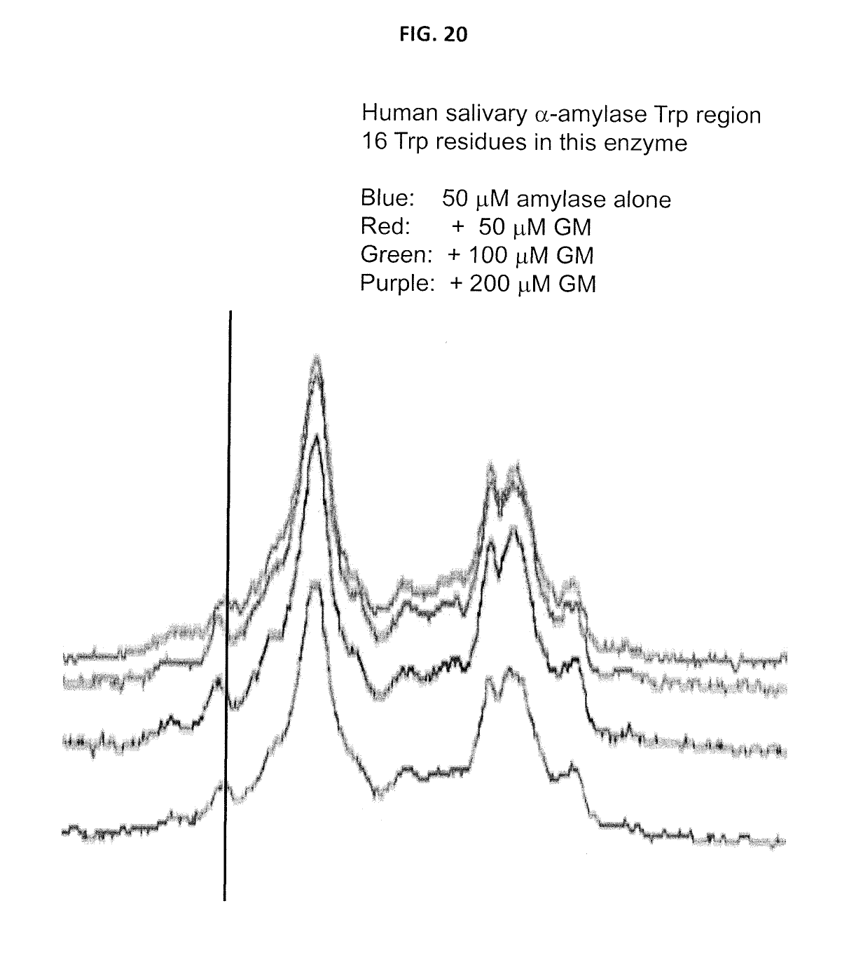

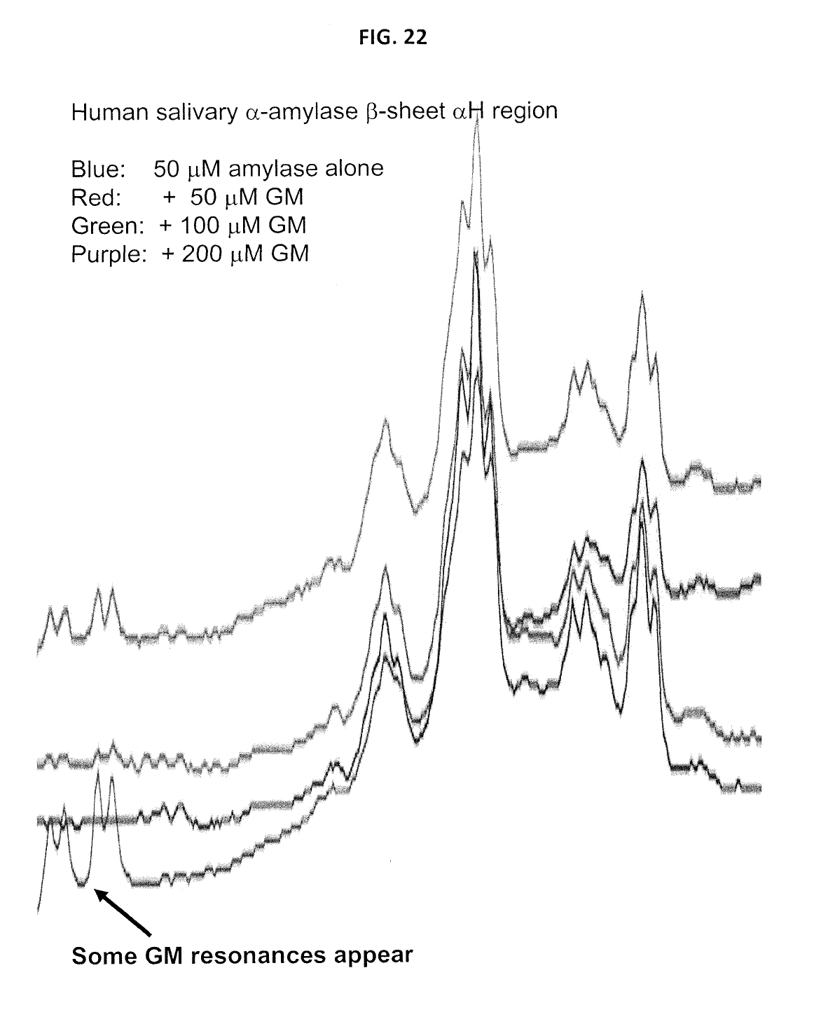

These NMR data as shown in FIGS. 20, 21 and 22 indicate that GM 1 indeed binds to the both human salivary amylase and porcine pancreatic amylase. The first four slides in this presentation show a few regions from the NMR spectra with HSA, where resonance changes are observed.

Because these amylases are relatively large, i.e. about 60 kDa, we only expected to see select changes to the NMR spectra, as observed. In fact, because most amylase resonances remain unperturbed in the presence of GM 1, we can also conclude that GM 1 does not greatly change the overall folded structure of either amylase.

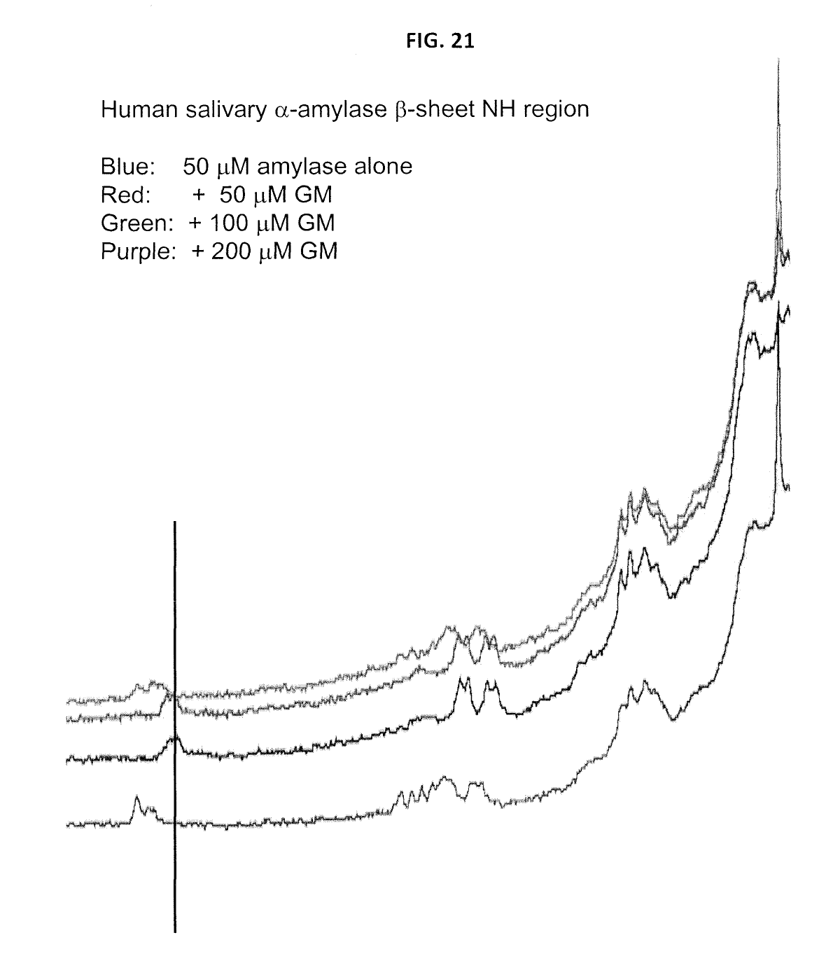

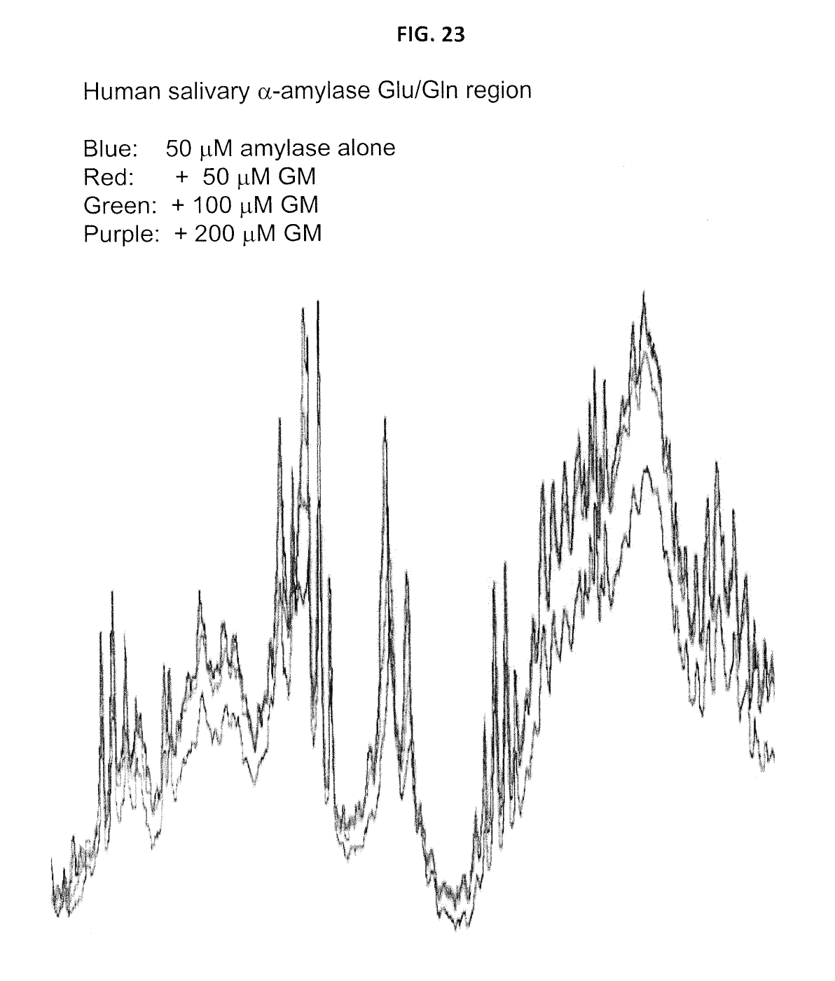

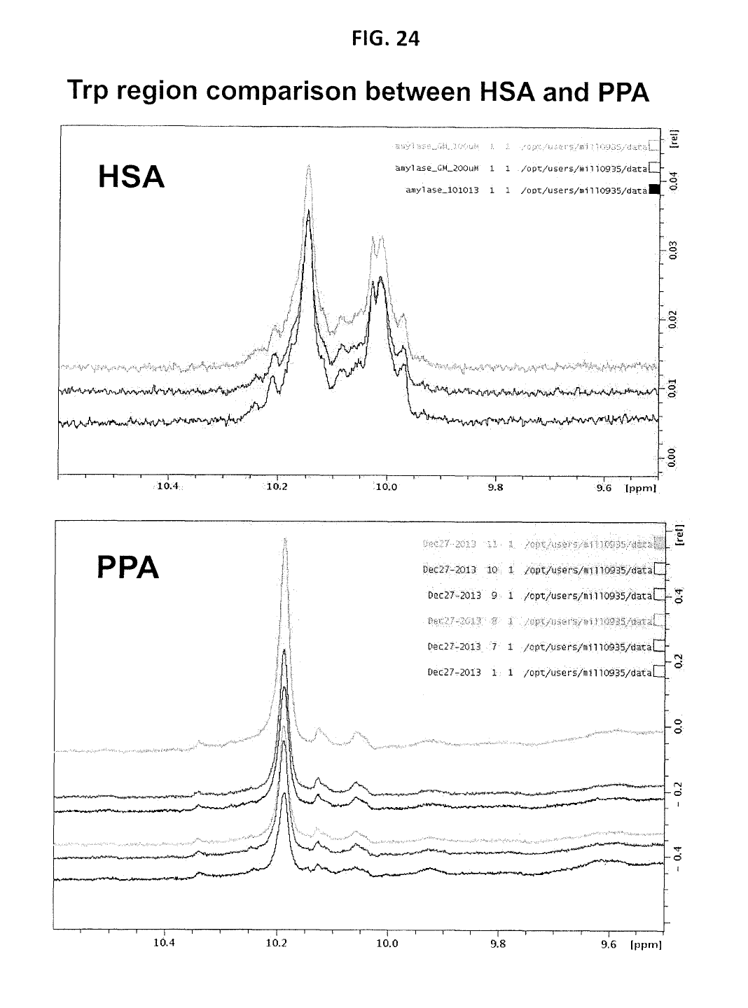

To elaborate somewhat on these slides, FIG. 20 shows the tryptophan (Trp) region of HAS with its 16 Trp residues. Note that most remain unperturbed in the presence of GM 1, but one in particular is perturbed. The same can be said with part of backbone NH region as shown in FIG. 21, as well as that of part of the β-sheet αH region as shown in FIG. 23 and side chain region of glutamic acid (Glu) and glutamine (Gln) residues as shown in FIG. 24. The X-ray structures of PPA and HPA are shown overlaid in slide 5 with the structure of bound Acarbose.

Note that this is the active site of the enzyme, whose β-sheet contains a key Trp and Gln residues. It is unknown whether GM I interacts at the same site, but it is intriguing to speculate that this is the case.

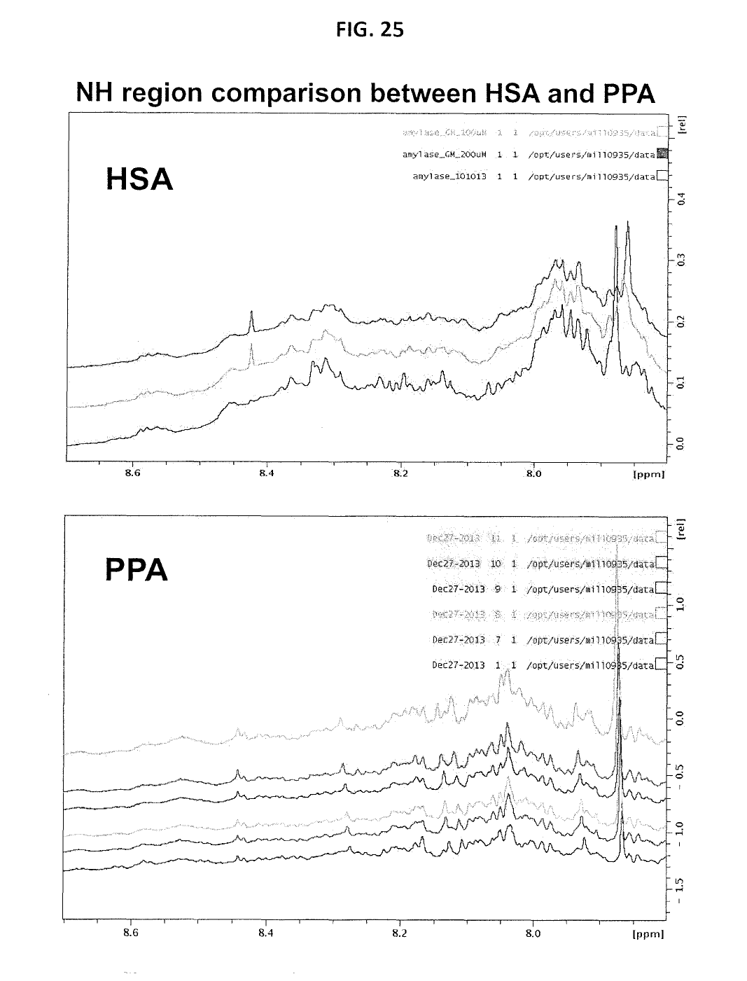

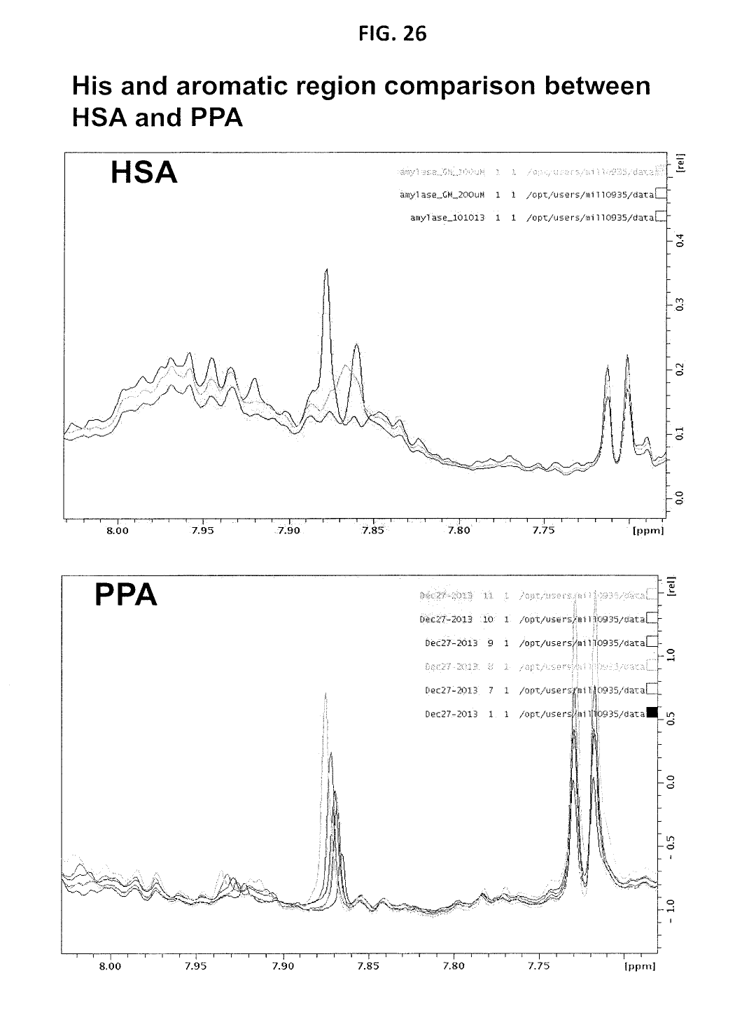

FIGS. 24 through 26 show some NMR data with PPA. Although the spectra of HSA and PPA look different, this is not unexpected. Nonetheless, conclusions made above with HSA are the same: I) GM I binds to PPA, 2) binding is selective, and 3) major perturbations to the structure of PPA do not occur. Analysis of these binding data suggests that Kci values fall in the μM range, perhaps about 50 μM, with somewhat stronger binding to PPA than to HSA. Materials & Methods

### Materials

Porcine pancreatic amylase was purchased from Megazyme, Inc. All other amylases, chemicals, and reagents were purchased from Sigma-Aldrich (St. Louis, Mo.), unless otherwise stated.

### Polysaccharide Preparations

PAZ320 components, GMα and GMP (mixed in molar ratio of 1:4), are hydrolyzed fractions of polysaccharides derived from fenugreek and guar gum, respectively, and both have weight average molecular weights of about 200 kDa (ref). GMa and GMP are primarily 1,4-P-o-galactomannans having Man/Gal ratios of 1.2 and 1.1, respectively.

### Solutions

All solutions, PPA and inhibitors were prepared in “amylase” buffer: 20 mM Potassium Phosphate, 2 μM CaCli, 10 μM DSS and 0.02% NaN3, pH 6.9. PPA was buffer exchanged 6 times using 1 OkDa Amicon Ultra-0.5 mL centrifugal filters. The filtrate was measured using the A280 program on a NanoDrop 8000 UV-Vis Spectrophotometer, diluted to 20 μM, aliquoted, and frozen until used. Human pancreatic amylase (HPA) was diluted with this buffer, measured, and used without further preparation.

Stock solutions for GMa and GMP were prepared by adding GMs to amylase buffer, vortexing for 15 min, and then incubating with shaking at RT 0/N. Starch/GM solutions were vortexed and either used immediately or incubated at RT overnight.

### Starch-Iodine Assay

The starch-iodine assay was modified from that reported by Xiao et al. (2006). A standard curve was constructed using 100 μL of 6 concentrations (0-1.0 mg/ml) and 100 μL of soluble starch. A total of 100 μL of 1 mg/ml of soluble starch+/−varying concentrations of GMs were incubated for 20 minutes at 30° C.+/−5 μL enzyme, for a total concentration of 1 μM PPA/well, and the absorbance read at 520 nm.

### NMR Spectroscopy

NMR experiments were carried out at 300 K on Bruker Avance 700 MHz or 850 MHz spectrometers equipped with a H/C/N triple-resonance probe and x/y/z triple-axis pulse field gradient unit. Conventional ¹H NMR experiments were carried out with a sweep width of 15 ppm A gradient sensitivity-enhanced version of two-dimensional ¹H-¹³C HSQC was also used, with 408 (tl)×2048 (t2) complex data points in carbon and proton dimensions, respectively.

NMR samples contained 600 μL of either 1 mg/ml soluble starch and/or maltohexaose and 10% D₂0+/−GMs or acarbose. NMR spectra were acquired prior to addition of enzyme, and then HPA or PPA was added directly to the NMR tube to a concentration of 1 μM, and consecutive NMR spectra were acquired as a function of time. Raw data were processed using NMRPipe (Delaglio et al., 1995) and were analyzed by using NMR view (Johnson and Blevins, 1994). Peak intensities were measured, and concentrations were determined by using a standard curve determined for maltose at known concentrations.

### Viscosity Measurements

Each mixture of GM+/−starch (15 ml) was prepared from concentrated stock solutions using “amylase” buffer (see above). Each sample was centrifuged for 5 minutes at 5000 rpm to remove airbubbles. Viscosity measurements were performed with a TA Instruments AR-G2 rheometer fitted with a concentric cylinder using bob and cup geometry at room temperature. Strain and frequency sweeps were initially performed on samples to define the linear viscoelastic region. Subsequent oscillatory stepped flow procedures were then undertaken with shear rate (1/s) ramped from 1 to 100, 100 to 0.01 and finally 1 to 1000. Results were visualized using Rheology Advantage Data Analysis software.

TABLE 1
Rates of amylase-mediated hydrolysis of starch as measured by the
starch-iodine assay and MT2/MT3 resonance intensity using NMR.
Amylase-inhibitory potential to assess the effectiveness of GMα and
GMβ on starch hydrolysis. Assays were run at 0.5, 1, 2, and 4 mg/mL;
acarbose at 400 μM, and starch at 1 mg/ml. U/ml = (As62
control − As62 sample)/(As62 starch × 20 min × 0.1 ml reaction
volume), and is interpreted to indicate X. U/ml values shown are
averages of 4 or 5 separate experiments done each in triplicate.

| | Cone | U/ml | Fraction | NMR rate | Fraction |
|---|---|---|---|---|---|
| | (mg/ml) | (mg/min) | Inhibited | (s⁻¹) | Inhibited |
| Starch | 1.0 | 1.86 ± 0.11 | | 29 | |
| PAZ320 | 2.5 | 1.02 ± 0.1 | 0.45 | | |
| GMα | 0.25 | | | | |
| | 0.5 | 1.3 ± 0.26 | 0.3 | 27 | 0.06 |
| | 1.0 | 1.2 ± 0.16 | 0.35 | 22 | 0.27 |
| | 2.0 | 0.72 ± 0.19 | 0.61 | 15 | 0.48 |
| | 4.0 | | | 7 | 0.75 |
| GMβ | 0.25 | | | | |
| | 0.5 | 1.73 ± 0.69 | 0.07 | | |
| | 1.0 | 1.85 ± 0.58 | 0.005 | | |
| | 2.0 | 1.68 ± 0.3 | 0.09 | | |
| | 4.0 | 1.54 ± 0.47 | 0.17 | | |
| | 8.0 | | | | |
| | 12.0 | | | | |
| | 16.0 | 0.76 ± 0.17 | 0.59 | | |
| Acarbose | | 0.38 ± 0.09 | 0.79 | | |
| Starch | 5.0 | 1.45 ± 0.11 | | | |
| GMα | 0.25 | 1.1 ± 0.11 | 0.27 | | |
| | 0.5 | 0.81 ± 0.26 | 0.44 | | |
| | 1.0 | 0.74 ± 0.16 | 0.49 | | |
| | 2.0 | 0.53 ± 0.19 | 0.64 | | |
| | 3.0 | 0.31 ± 0.19 | 0.78 | | |

Although exemplary embodiments have been presented in order to further elucidate these teachings, it should be noted that these teachings are not limited only to those exemplary embodiment.

Although the invention has been described with respect to various embodiments, it should be realized these teachings are also capable of a wide variety of further and other embodiments within the spirit and scope of the appended claims.

The specification is most thoroughly understood in light of the teachings of the references cited within the specification. The embodiments within the specification provide an illustration of embodiments of the invention and should not be construed to limit the scope of the invention. One skilled in the art readily recognizes that many other embodiments are encompassed by the invention. Those skilled in the art will recognize, or be able to ascertain using no more than routine experimentation, many equivalents to the specific embodiments of the invention described herein. Such equivalents are intended to be encompassed by the following appended claims.

What is claimed:

1. A method for inhibiting the action of hydrolyzing enzymes that break down carbohydrates, thereby diminishing the release of glucose into the bloodstream comprising,
administering a composition having at least one first purified soluble mannan polysaccharide of high molecular weight and a second purified mannan polysaccharide of low molecular weight in combination with at least one oligosaccharide and/or monosaccharide.

2. The method according to claim 1, wherein the composition has at least one first purified soluble mannan polysaccharide of high molecular is about 50 kD to about 300 kD.

3. The method according to claim 1, wherein the composition has at least one second purified mannan polysaccharide of low molecular weight is about 5 kD to about 50 kD.

4. The method according to claim 1, wherein the composition has a ratio of low molecular weight mannan polysaccharide to high molecular weight mannan polysaccharide is about 2:1 to about 100:1 by weight.

5. The method according to claim 1, wherein the composition has at least one of the at least one first purified soluble mannan polysaccharide of high molecular weight and the at least one second purified mannan polysaccharide of low molecular weight is fractionated from one or more legume seeds, the one or more legume seeds including at least one of Cassia fistula, Ceratonia siliqua, Caesalpinia spinosa Trigonelle foenumgraecum, and/or Cyamopsis tetragonolobus.

6. The method according to claim 1, wherein the composition has at least one first purified soluble mannan polysaccharide of high molecular weight and the at least one second purified mannan polysaccharide of low molecular weight are purified to at least about 90% polymeric carbohydrates.

7. The method according to claim 1, wherein the composition has at least one first purified soluble mannan polysaccharide of high molecular weight and the at least one second purified mannan polysaccharide of low molecular weight are purified to contained less than about 1% of natural non-polysaccharides impurities including proteins, alkaloids, glycoalkaloids and provide hypoallergenic dietary fibers.

8. The method according to claim 1, wherein the composition has at least one first purified soluble mannan polysaccharide of high molecular weight and the at least one second purified mannan polysaccharide of low molecular weight are purified to remove at least a portion of environmental and agricultural contaminants including heavy metals, pesticides, herbicides, microbial toxins and mycotoxins.

9. The method according to claim 1, wherein the composition has at least one first purified soluble mannan polysaccharide of high molecular weight is embedded in the at least one second purified mannan polysaccharide of low molecular weight forming at least one combined mannan polysaccharide; and wherein the at least one combined mannan polysaccharide is embedded in the at least one oligosaccharide and/or monosaccharide.

10. The method according to claim 1, wherein the composition contains:
about 1% to about 25% (wt/wt) of the at least one first purified soluble mannan polysaccharide of high molecular weight, about 20% to about 80% (wt/wt) of the at least one second purified mannan polysaccharide of low molecular weight, and
about 40% to about 60% (wt/wt) of the at least one oligosaccharide
and/or monosaccharide.
